(19) United States
(12) Patent Application Publication
(10) Pub. No.: US 2022/0113288 A1
(43) Pub. Date: Apr. 14, 2022

(54) NEUROTRANSMITTER-BASED BRAIN MAPPING METHOD AND USE OF BRAIN MAP

(71) Applicant: Neurovis Inc., Chungcheongnam-do (KR)

(72) Inventors: Hyung Gun Kim, Seoul (KR); Sung Hyun Hong, Seoul (KR); Guk Hwa Jung, Chungcheongnam-do (KR); Hye Ran Park, Chungcheongnam-do (KR); Kee Chan Ahn, Chungcheongnam-do (KR); Hak Rim Kim, Seoul (KR)

(21) Appl. No.: 17/430,263

(22) PCT Filed: Feb. 12, 2020
(86) PCT No.: PCT/KR2020/001988

§ 371 Date: Aug. 11, 2021

(30) Foreign Application Priority Data
Feb. 12, 2019 (KR) 10-2019-0016158
Feb. 12, 2020 (KR) 10-2020-0017218

(51) Int. Cl. G01N 30/72; G01N 33/50

(52) U.S. Cl. CPC G01N 30/72; G01N 2030/027; G01N 2560/00; G01N 33/5008

(57) ABSTRACT

An embodiment pertains to a method for evaluating efficacy of a drug which increases or decreases the secretion of a particular neurotransmitter, by measuring a concentration change of the particular neurotransmitter in a specific intracerebral site with reference to a brain map, the method comprising the steps of: selecting as a microdialysis target region in the brain map a first site of an animal, which corresponds to a site that the brain map represents as being the highest in the concentration of a first neurotransmitter of which the secretion is increased or decreased by the drug; and injecting the drug to the animal and monitoring a concentration change of the first neurotransmitter in the first site between pre- and post-injection of the drug. The brain map is constructed by acquiring a concentration distribution of 11 or more multiple neurotransmitters including serotonin, dopamine, GABA, glutamate, and metabolites thereof, obtained by mass analysis of samples acquired from multiple sites in the human brain—hereinafter referred to as first concentration distribution—and a concentration distribution of 11 or more multiple neurotransmitters including serotonin, dopamine, GABA, glutamate, and metabolites thereof, obtained by mass analysis of samples acquired from multiple sites in a monkey brain—hereinafter referred to as second concentration distribution—, and utilizing first correlation including at least 11 correlation data resulting from matching the multiple sites of the human brain to the multiple sites of the monkey brain on the basis of similarity in the concentration distribution of the individual neurotransmitters between the first concentration distribution acquired and the second concentration distribution acquired, and the second concentration distribution, wherein the first site corresponds to a second site in the second concentration distribution when the first neurotransmitter is the most abundant at the second site in the first concentration distribution.

### TECHNICAL FIELD

The present invention relates to a method for making a brain map using an intracerebral neurotransmitter and utilizing the brain map, and more particularly, to a neurotransmitter-based brain map which is make by measuring the concentration of each of various neurotransmitters for each region in the brain and measuring a change in a concentration of the neurotransmitter, and the like in a region where a specific neurotransmitter is distributed at a high concentration using the make brain map.

### BACKGROUND ART

Mental disorders, pain and drug addiction are phenomena manifested by abnormalities in neurotransmitters in the central nervous system. Recently, at a preclinical phase for animals, attempts have been made to develop drugs that are effective for mental disorders, pain and drug addiction by measuring the concentration of neurotransmitters which act in the central nervous system, and accordingly, there is a growing trend of CRO (Contract Research Organization) companies performing the preclinical phase.

Meanwhile, recently, with the development of a brain map make technique such as the Allen Brain Map, in which researchers commonly participate, interest in the brain has increased and research thereon is actively being conducted. However, the Allen Brain Map has a problem in that it is difficult to understand a mechanism by which neurotransmitters are actually generated and act because a brain map is made for the brain based on genes, mRNA, and the like.

In order to overcome the problem, there is a need for make of brain maps of experimental animals having a neurotransmitter concentration distribution, which is similar to that of the human brain at a preclinical phase for understanding the brain based on the neurotransmitters and evaluating a drug, and a neurotransmitter analysis technique using the same.

### DISCLOSURE

### Technical Problem

An object of the present invention is to provide a method for make a brain map in order to grasp a neurotransmitter concentration distribution.

Another object of the present invention is to provide a position for obtaining a sample for measuring a neurotransmitter from an experimental animal based on the neurotransmitter concentration distribution of the experimental animal.

Still another object of the present invention is to provide a position for obtaining a sample from an experimental animal based on the similarity of neurotransmitter concentrations in the brain of the experimental animal and the brain of a human.

Yet another object of the present invention is to provide a method for fixing a microdialysis probe when a microdialysis method is used.

The objects to be achieved by the present invention are not limited to the above-described objects, and other objects that have not been mentioned will be clearly understood by a person with ordinary skill in the art to which the present invention pertains from the present specification and the accompanying drawings.

### Technical Solution

An embodiment may provide a method for evaluating the efficacy of a drug capable of increasing or decreasing a specific neurotransmitter, wherein the method measures a change in a concentration of the specific neurotransmitter in a specific region of a brain as an evaluation index by referring to a brain map, the method comprises: selecting a first region in an animal brain as a microdialysis target region; wherein the first region corresponds to a region indicated in the brain map, where a concentration of a first neurotransmitter being increased or decreased by the drug is highest; and injecting the drug into the animal and confirming a change in the concentration of the first neurotransmitter at the first region before and after the injection of the drug, respectively; wherein, the brain map is prepared by the following: obtaining a concentration distribution for a plurality of 11 or more neurotransmitters including serotonin, dopamine, GABA, glutamate, and metabolites thereof, which is obtained by performing mass spectrometry on a sample taken from a plurality of regions of an extracted human brain (hereinafter, referred to as a first concentration distribution), and a distribution for a plurality of 11 or more neurotransmitters including serotonin, dopamine, GABA, glutamate, and metabolites thereof, which is obtained by performing mass spectrometry on a sample taken from a plurality of regions of a monkey brain (hereinafter, referred to as a second concentration distribution), making the brain map using a first correlation and the second concentration distribution, wherein the first correlation includes at least 11 pieces of correlation data in which the plurality of regions of the human brain correspond to the plurality of regions of the monkey brain based on the similarity of the concentration distributions of the individual neurotransmitter, when the amount of the first neurotransmitter in the first concentration distribution is highest, a corresponding region is determined as a second region, and a region in the second concentration distribution, which corresponds to the second region is determined as the first region.

Another embodiment may provide a method for evaluating the efficacy of a drug capable of increasing or decreasing a specific neurotransmitter by referring to a brain map, wherein the method measures a change in a concentration of the specific neurotransmitter in a specific region of a brain as an evaluation index by referring to a brain map, and the specific region in the brain is a position in the animal brain showing a difference in the concentration distribution of the specific neurotransmitter in an animal with any disease (hereinafter, referred to as a disease model) and an animal without any disease (hereinafter, referred to as a normal model), the method comprising: selecting a first region as a microdialysis target region using the brain map; wherein the concentrations of a first neurotransmitter in the disease model and the normal model differ by a predetermined level or more; and injecting the drug into the animal other than a human and confirming a change in the concentration of the first neurotransmitter in the first region before and after the injection of the drug, respectively; wherein, the brain map is prepared by the following: obtaining a concentration distribution for a plurality of 11 or more neurotransmitters including serotonin, dopamine, GABA, glutamate, and metabolites thereof, which is obtained by performing mass spectrometry on a sample taken from a plurality of regions of a human brain (hereinafter, referred to as a first concentration distribution), and a concentration distribution for a plurality of 11 or more neurotransmitters including serotonin, dopamine, GABA, glutamate, and metabolites thereof, which is obtained by performing mass spectrometry on a sample taken from a plurality of regions of a monkey brain (hereinafter, referred to as a second concentration distribution), making the brain map using a first correlation and the second concentration distribution, wherein the first correlation includes at least 11 or more pieces of correlation data in which the plurality of regions of the human brain correspond to the plurality of regions of the monkey brain based on the similarity of the concentration distributions of the individual neurotransmitter confirmed in the obtained first concentration distribution and the obtained second concentration distribution, wherein the step of selecting of the first region selects the first region to be a region corresponding to the second region and a third region in the second concentration distribution when the first concentration distribution in a second region of the brain of a human with any disease and the first concentration distribution in the third region which is a region anatomically identical to the second region of the brain of the human without any disease have a difference in concentration, which is a predetermined level or more, for the first neurotransmitter among the plurality of neurotransmitters by comparing the first concentration distribution of the human with any disease with the first concentration distribution of the human without any disease.

Still another embodiment may provide a method for fixing a microdialysis probe in order to obtain a microdialysis sample from an animal brain, the method comprising: placing a mesh on a surface of the brain, wherein the mesh is to prevent the microdialysis probe from falling off without being fixed by removing mixture on the surface of the brain; inserting a microdialysis probe into at least one of a plurality of cavities of the mesh such that the mesh comprises a wire dividing the surface of the brain into a predetermined size and the plurality of cavities generated according to the division, and at least a portion of the microdialysis probe is inserted into a monkey brain; and adhering the microdialysis probe to a predetermined part including a position where the microdialysis probe is inserted into the mesh and the surface of the brain using an adhesive in order to fix the microdialysis probe to the surface of the animal brain, wherein the step of adhering the microdialysis comprises subsequently placing a first adhesive for reducing the moisture of a position where the microdialysis probe is inserted and a second adhesive for firmly fixing the microdialysis probe.

The means for solving the problems of the present invention are not limited to the above-described means, and the means for solving the problems that have not been mentioned will be clearly understood by a person with ordinary skill in the art to which the present invention pertains from the present specification and the accompanying drawings.

### Advantageous Effects

According to the present invention, it is possible to make a brain map for grasping the concentration of a neurotransmitter.

According to the present invention, positions in the brain of an experimental animal and the brain of a human for measuring a neurotransmitter can be provided.

According to the present invention, a microdialysis probe can be firmly fixed on a tissue during microdialysis.

The effects of the present invention are not limited to the above-described effects, and other effects that have not been mentioned will be clearly understood by a person with ordinary skill in the art to which the present invention pertains from the present specification and the accompanying drawings.

### DESCRIPTION OF DRAWINGS

FIG. 1 is a view illustrating a brain map in which the concentration of a neurotransmitter is indicated for each position in the brain of a rhesus monkey according to an embodiment.

FIG. 2 is a view illustrating the brains of a mouse among rodents, a rhesus monkey among primates, and a human according to an embodiment.

FIG. 3 is a view showing that a microdialysis probe was inserted into a cortical region of the brain of an animal and then fixed using an adhesive in order to acquire a sample according to an embodiment.

FIG. 4 is a schematic view showing that when a microdialysis probe is fixed on a tissue according to an embodiment, the microdialysis probe is fixed using a mesh.

FIG. 5(a) is a schematic view illustrating a brain map in which the concentration of a neurotransmitter is illustrated at each position of the brain according to an embodiment.

FIG. 5(b) is a graph showing the concentration of a neurotransmitter (dopamine) at each position of the brain according to an embodiment.

FIG. 6 is a flow chart for explaining a method for measuring the concentration of a neurotransmitter using a brain map in order to evaluate the efficacy of a drug in a target animal according to an embodiment.

FIG. 7 is a flow chart for explaining a method for measuring the concentration of a neurotransmitter in a region of a target animal brain where the concentration of the neurotransmitter is highest, using a brain map in order to evaluate the efficacy of a drug in the target animal according to an embodiment.

FIG. 8 is a flow chart for explaining a method for measuring the concentration of a neurotransmitter in a region of a target animal brain, which matches a region where the concentration of a neurotransmitter to be evaluated for drug efficacy in the human brain is highest, using a brain map in order to evaluate the efficacy of a drug in the target animal according to an embodiment.

FIG. 9 is a flow chart for explaining a method for measuring the concentration of a neurotransmitter in regions where the concentrations of the neurotransmitter are different by comparing a disease model with a normal model in a target animal, using a brain map in order to evaluate the efficacy of a drug in the target animal according to an embodiment.

FIG. 10 is a flow chart for explaining a method for measuring the concentrations of a neurotransmitter in a region in a target animal brain, which matches regions where the concentrations of the neurotransmitter are different by comparing a disease model human with a normal model human, using a brain map in order to evaluate the efficacy of a drug in the target animal according to an embodiment.

FIG. 11 is a flow chart for showing that a brain map is made based on neurotransmitters according to an embodiment.

FIGS. 12 and 13 are views showing that the human brain is cut into even sizes according to an embodiment.

FIG. 14 is a view showing that a sample is obtained by punching a section of the brain according to an embodiment.

FIG. 15 is a schematic view showing that a human brain map and a monkey brain map according to an embodiment are matched based on the similarity in neurotransmitter concentrations.

FIG. 16 is a view illustrating the anatomically identical positions of the human brain and the monkey brain according to an embodiment.

FIGS. 17 to 38 illustrate the concentration of each neurotransmitter at a plurality of positions in a primate brain according to an embodiment.

FIG. 39 is a graph showing the regions where samples can be obtained from the primate brain using the concentration distribution of GABA according to an embodiment.

FIG. 40 is a graph showing the regions where samples are obtained for simultaneously analyzing GABA and glutamate in the primate brain according to an embodiment.

FIG. 41 is a graph showing the regions where samples are obtained for simultaneous analyzing dopamine and MHPG-sulfate from the primate brain according to an embodiment.

FIGS. 42 to 52 illustrates the concentration of each neurotransmitter in a plurality of positions in the human brain according to an embodiment.

FIG. 53 is a graph showing the concentration of GABA measured in a plurality of regions of the primate brain and the human brain according to an embodiment.

FIG. 54 is a graph showing the concentration of GABA measured in a plurality of regions of the primate brain and the human brain according to an embodiment.

FIGS. 55 to 58 illustrate the results of analyzing neurotransmitters for samples of the primate brain according to an embodiment.

### MODES OF THE INVENTION

The above-described objects, features and advantages of the present invention will become more apparent through the following detailed description associated with the accompanying drawings. However, since the present invention may be modified into various forms and include various exemplary embodiments, hereinafter, specific exemplary embodiments will be illustrated in the drawings and described in detail.

In the drawings, the thicknesses of layers and regions are exaggerated for clarity, and further, an element or layer referred to as being “on” another element or layer includes not only a case where the element or layer is situated directly on another element or layer, but also a case where another layer or another element is interposed therebetween. Throughout the specification, like reference numerals denote like elements in principle. In addition, elements having the same function within the scope of the same idea shown in the drawings of each embodiment will be described using the same reference numerals.

When detailed descriptions on known functions or configurations related to the present invention are determined to unnecessarily obscure the gist of the present invention, the detailed description will be omitted. Furthermore, numbers (for example, first, second, and the like) used in the description of the present specification are just identification symbols to distinguish one element from another element.

Further, the suffixes “module” and “part” for elements used in the following description are given or mixed in consideration of the ease of specification, and do not have distinct meanings or roles by themselves.

An embodiment of the present invention may provide a method for evaluating the efficacy of a drug capable of increasing or decreasing a specific neurotransmitter, wherein the method measures a change in a concentration of the specific neurotransmitter in a specific region of a brain as an evaluation index by referring to a brain map, the method comprises: selecting a first region in an animal brain as a microdialysis target region; wherein the first region corresponds to a region indicated in the brain map, where a concentration of a first neurotransmitter being increased or decreased by the drug is highest; and injecting the drug into the animal and confirming a change in the concentration of the first neurotransmitter at the first region before and after the injection of the drug, respectively; wherein, the brain map is prepared by the following: obtaining a concentration distribution for a plurality of 11 or more neurotransmitters including serotonin, dopamine, GABA, glutamate, and metabolites thereof, which is obtained by performing mass spectrometry on a sample taken from a plurality of regions of an extracted human brain (hereinafter, referred to as a first concentration distribution), and a distribution for a plurality of 11 or more neurotransmitters including serotonin, dopamine, GABA, glutamate, and metabolites thereof, which is obtained by performing mass spectrometry on a sample taken from a plurality of regions of monkey brain (hereinafter, referred to as a second concentration distribution), making the brain map using a first correlation and the second concentration distribution, wherein the first correlation includes at least 11 pieces of correlation data in which the plurality of regions of the human brain correspond to the plurality of regions of the monkey brain based on the similarity of the concentration distributions of the individual neurotransmitter, when the amount of the first neurotransmitter in the first concentration distribution is highest, a corresponding region is determined as a second region, and a region in the second concentration distribution, which corresponds to the second region is determined as the first region.

Another embodiment of the present invention may provide a method for evaluating the efficacy of a drug capable of increasing or decreasing a specific neurotransmitter by referring to a brain map, wherein the method measures a change in a concentration of the specific neurotransmitter in a specific region of a brain as an evaluation index by referring to a brain map, and the specific region in the brain is a position in the animal brain showing a difference in the concentration distribution of the specific neurotransmitter in an animal with any disease (hereinafter, referred to as a disease model) and an animal without any disease (hereinafter, referred to as a normal model), the method comprising: selecting a first region as a microdialysis target region using the brain map; wherein the concentrations of a first neurotransmitter in the disease model and the normal model differ by a predetermined level or more; and injecting the drug into the animal other than a human and confirming a change in the concentration of the first neurotransmitter at the first region before and after the injection of the drug, respectively; wherein, the brain map is prepared by the following: obtaining a concentration distribution for a plurality of 11 or more neurotransmitters including serotonin, dopamine, GABA, glutamate, and metabolites thereof, which is obtained by performing mass spectrometry on a sample taken from a plurality of regions of a human brain (hereinafter, referred to as a first concentration distribution), and a concentration distribution for a plurality of 11 or more neurotransmitters including serotonin, dopamine, GABA, glutamate, and metabolites thereof, which is obtained by performing mass spectrometry on a sample taken from a plurality of regions of a monkey brain (hereinafter, referred to as a second concentration distribution), making the brain map using a first correlation and the second concentration distribution, wherein the first correlation includes at least 11 or more pieces of correlation data in which the plurality of regions of the human brain correspond to the plurality of regions of the monkey brain based on the similarity of the concentration distributions of the individual neurotransmitter confirmed in the obtained first concentration distribution and the obtained second concentration distribution, wherein the step of selecting of the first region selects the first region to be a region corresponding to the second region and a third region in the second concentration distribution when the first concentration distribution in a second region of the brain of the human with any disease and the first concentration distribution in the third region which is a region anatomically identical to the second region of the brain of the human without any disease have a difference in concentration, which is a predetermined level or more, for the first neurotransmitter among the plurality of neurotransmitters by comparing the first concentration distribution of the human with any disease with the first concentration distribution of the human without any disease.

Still another embodiment of the present invention may provide a method for fixing a microdialysis probe in order to obtain a microdialysis sample from an animal brain, the method comprising: placing a mesh on a surface of the brain, wherein the mesh is to prevent the microdialysis probe from falling off without being fixed by removing moisture on the surface of the brain; inserting a microdialysis probe into at least one of a plurality of cavities of the mesh such that the mesh comprises a wire dividing the surface of the brain into a predetermined size and the plurality of cavities generated according to the division, and at least a portion of the microdialysis probe is inserted into a monkey brain; and adhering the microdialysis probe to a predetermined part including a position where the microdialysis probe is inserted into the mesh and the surface of the brain using an adhesive in order to fix the microdialysis probe to the surface of the animal brain, wherein the step of adhering the microdialysis comprises subsequently placing a first adhesive for reducing the moisture of a position where the microdialysis probe is inserted and a second adhesive for firmly fixing the microdialysis probe.

1 Preparation for Analysis of Neurotransmitters Using Brain Map

1.1 Overview

Hereinafter, a method for measuring the concentration of a neurotransmitter will be described using a brain map according to an embodiment of the present invention.

The brain map for measuring the concentration of the neurotransmitter according to an embodiment of the present invention may be a brain map made by measuring the concentration of the neurotransmitter at each position of the brain. However, the brain map for measuring the concentration of the neurotransmitter is not limited thereto, and may be a brain map made by measuring the concentration of a neurotransmitter receptor, the protein expression level according to the metabolism of the neurotransmitter and the concentration of mRNA.

Further, the brain map for measuring the concentration of the neurotransmitter according to an embodiment may be a brain map in which the concentration for one specific neurotransmitter is indicated for each position in the brain, and may be a brain map in which the concentrations for a plurality of neurotransmitters are indicated for each position. The brain map in which the concentration of the neurotransmitter is indicated will be described in more detail in the following brain map make section.

However, the brain map for measuring the concentration of the neurotransmitter in the present specification is not limited to the above-described examples, and it is needless to say that a brain map capable of indicating the activation degree of the neurotransmitter at each position of the brain can also be used as a brain map for measuring the concentration of the neurotransmitter described in the present specification.

1.2 Types of Neurotransmitters Indicated on Brain Map

The brain map can indicate the concentration of a plurality of neurotransmitters. Here, the plurality of neurotransmitters may be neurotransmitters selected from at least amino acid-based neurotransmitters, acetylcholine-based neurotransmitters, monoamine-based neurotransmitter, trace amine-based neurotransmitters, lipid-based neurotransmitter, purine-based neurotransmitters and opioid-based neurotransmitters.

Specifically, the amino acid-based neurotransmitter may include arginine, aspartate, glutamate, gamma-aminobutyric acid (hereinafter, referred to as GABA), glycine and D-serine.

In addition, the acetylcholine-based neurotransmitter may include acetylcholine.

Furthermore, the monoamine-based neurotransmitter may include dopamine, norepinephrine, epinephrine, serotonin (5-hydroxytryptamine) and histamine.

Further, the trace amine-based neurotransmitter may include tyramine, octopamine, synephrine, tryptamine and N-methyltryptamine.

In addition, the lipid-based neurotransmitter may include anandamide, 2-arachidonoylglycerol, 2-arachidonyl glyceryl ether, N-arachidonoyl dopamine and virodhamine.

Furthermore, the purine-based neurotransmitter may include adenosine, adenosine triphosphate and nicotinamide adenine dinucleotide.

Further, the opioid-based neurotransmitter may include encephalin, dynorphin, endorphin, endomorphin and nociceptin/orphanin FQ.

In addition, a neurotransmitter such as 2-phenylalanine, glutamine, 5-HIAA, MHPG-sulfate, choline, dopamine, DOPAC, HVA, 3-MT, substance P, beta-endorphine, Met-enkephalin, Leu-enkephalin, dynorphin A, agmatine, spermine, spermidine, and putrescine and metabolites thereof may be additionally included among the neurotransmitters.

However, the types of neurotransmitters that can be indicated on the brain map are not limited to the neurotransmitter examples described above, and it is needless to say that metabolites metabolized based on the above-described neurotransmitter and all chemical materials used as mediators to deliver proteins and neural signals may be included among the types of neurotransmitters indicated on the brain map.

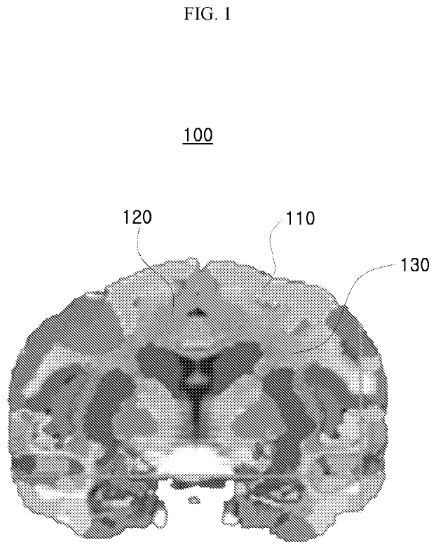

FIG. 1 is a view illustrating a brain map in which the concentration of a neurotransmitter is indicated for each position in the brain of a rhesus monkey according to an embodiment.

According to an embodiment, referring to FIG. 1, the brain map can exhibit a position where each neurotransmitter is present at high concentrations. For example, a first region (110 in the FIG. 1) may exhibit a position where glutamate is present at the highest concentration, and a second region (120 in the FIG. 1) may exhibit a position where dopamine is present at the highest concentration. Here, the first region and the second region do not refer to specific regions for each part of the brain, but indicate positions where different neurotransmitters are present at high concentrations.

In this case, a third region (130 in the FIG. 1) may be a region in which the concentration of a specific neurotransmitter is not high because the concentration of each neurotransmitter is simultaneously high or low, or may be a region in which the concentration of the neurotransmitter is not measured.

According to another embodiment, referring to FIG. 1, the brain map can exhibit the concentration of one neurotransmitter for each position. For example, when the relative concentration of glutamate has a value from 0 to 1, the first region (110) can exhibit a position where the glutamate has a concentration of 1, and the second region (120) can exhibit a position wherein the glutamate has a concentration of 0.5. Furthermore, the third region (130) can exhibit a position where the glutamate has a concentration of 0. Here, the relative concentration is not limited to the value from 0 to 1, and various display values including molar concentration, mass concentration and % concentration for indicating the concentration can be used.

1.3 Target Animals for Sample Analysis

When an animal to become a preclinical target for evaluating the efficacy of a drug is selected, the results obtained in the preclinical phase and the results obtained in the clinical phase may differ from each other. This is because the human brain and the preclinical animal brain may be anatomically different.

Accordingly, in order to discover a drug that is effective in the clinical phase, an animal having a distribution similar to the distribution of neurotransmitters in the human brain may be used as the target animal. However, the target animal is not limited thereto, and it is possible to use an animal in which a mechanism of action of a drug in the human brain and a mechanism of action of a drug in the target animal brain are similar.

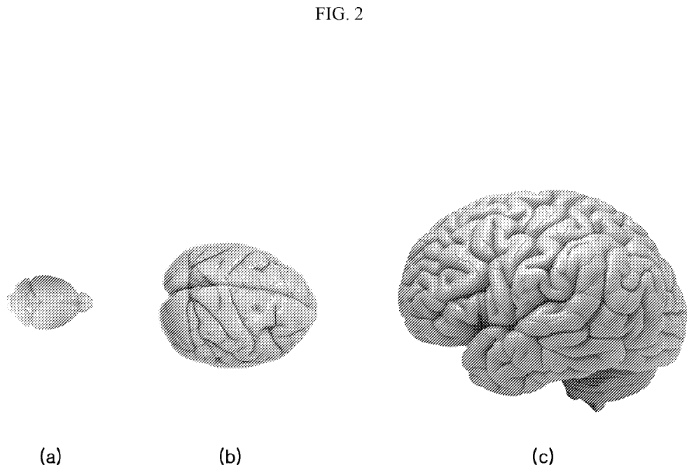

FIG. 2 is a view illustrating the brains of a mouse among rodents, a rhesus monkey among primates, and a human according to an embodiment.

Referring to FIG. 2, the brains of the rodent, primate and human may have anatomically different structures. However, the brains of the rodent, primate and/or human may have similar neurotransmitter concentration distributions.

That is, the human brain may have a specific region in which the concentration distribution of the neurotransmitter is similar to that of the target animal. In other words, in a specific region of the brain where the concentration distributions of the neurotransmitters in the target animal are similar, the human brain and the target animal brain may have similar neurotransmitter concentration distributions.

According to an embodiment, in a specific region of the brain with similar neurotransmitter concentration distributions, the human brain may exhibit a pattern similar to drug-induced neurotransmitter concentration changes in the target animal's brain. That is, changes in concentration of neurotransmitters in the human brain may be predicted on the basis of changes in the neurotransmitter concentration distribution in the target animal brain. Here, the target animal used may be the monkey's brain, which has the highest similarity to the human brain. However, the target animal is not limited thereto, and various target animals such rodents and birds having a distribution of neurotransmitters similar to that of the human brain may be used.

1.4 Method of Obtaining Sample for Analysis

In order to analyze the concentration of a neurotransmitter using the brain map, a sample for each part of the brain may be obtained. Typically, a sample for concentration analysis may be obtained using a microdialysis method in an in-vivo state.

Specifically, by the microdialysis method according to an embodiment, a sample for analysis may be obtained by inserting a microdialysis probe into a position from which a sample is to be obtained in a state in which an animal including a human is alive.

According to another embodiment, a method of obtaining a sample may be performed in an in-vitro state. A tissue for use as a sample may be obtained by tissue biopsy. A tissue obtained by biopsy may be ground and used as a sample. Alternatively, the sample may be obtained from the tissue obtained through biopsy by a microdialysis method.

Hereinafter, the above-described method of obtaining a sample will be described on the basis of a case where the microdialysis method is used in an in-vivo state. However, the obtaining of the sample using the microdialysis method described in the present specification may be similarly applied to a method of performing microdialysis in an in-vitro state from a tissue partially falling off the brain, and the like.

1.5 Method of Fixing Microdialysis Probe

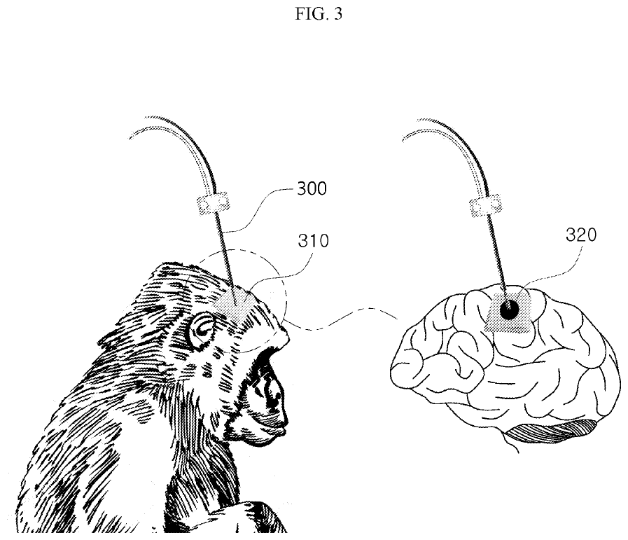

According to an embodiment, when a sample is taken using the microdialysis method, the sample may be taken using a microdialysis probe (300 in FIG. 3).

The microdialysis probe (300) may be inserted into a target region for obtaining a sample. Typically, a target region for obtaining a sample may be a cortical region of the brain. However, the target region for obtaining a sample is not limited thereto, and may be all body tissues including the animal brain or the human brain. Hereinafter, for convenience of description, a position where the microdialysis probe is inserted is referred to as the cortical region of the brain.

FIG. 3 is a view showing that a microdialysis probe was inserted into a cortical region of the brain of an animal and then fixed using an adhesive in order to acquire a sample according to an embodiment.

An adhesive (320 in the FIG. 3) can fix the microdialysis probe (300) on a tissue. For example, the adhesive (320) may be dental cement. In addition, the adhesive (320) may include all adhesive materials capable of adhering the microdialysis probe (300) on a body tissue, including bio-bonds.

According to an embodiment, referring to FIG. 3, when a sample is obtained using the microdialysis method, the microdialysis probe (300) can be adhered on a tissue (310 in the FIG. 3) using the adhesive (320). For example, when the tissue (310) from which a sample is to be obtained is in a cortical region of the brain, the microdialysis probe (300) is inserted into the cortical region of the brain, and then the adhesive (320) may be applied to a region where the microdialysis probe (300) is inserted. The adhesive (320) can fix the microdialysis probe (300). Here, the adhesive (320) may be a bio-adhesive capable of adhering the microdialysis probe (300).

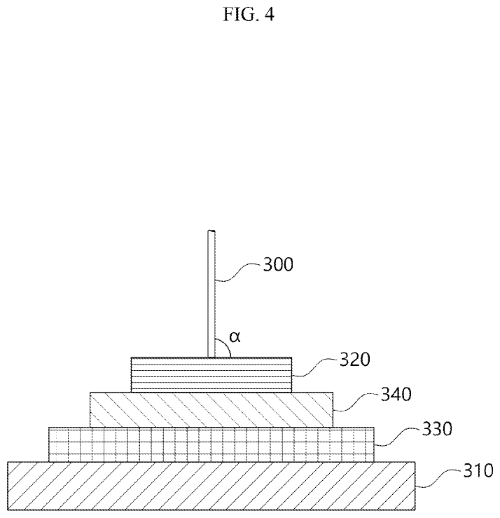

FIG. 4 is a schematic view showing that when a microdialysis probe is fixed on a tissue according to an embodiment, the microdialysis probe is fixed using a mesh.

A mesh (330 in the FIG. 4) may be a grid-shaped structure. Here, the grid shape may be a shape having a lattice structure as a specific region is uniformly partitioned. However, although the uniform partitioning here may mean that the partitioned size is constant, the partitioning is not limited thereto, and may include partitioning a specific region with a certain regularity. At this time, the partitioned size may be a size large enough for the microdialysis probe (300 in the FIG. 4) to be inserted.

The mesh (330) may be made of a material capable of absorbing moisture. For example, the mesh (330) may be absorbent cotton which is generally used. Here, the mesh (330) is not limited to the above-described example, and may be made of a fiber yarn capable of absorbing moisture.

An adhesive for removing moisture (340 in the FIG. 4) may be a bio-bond. However, the adhesive is not limited thereto, and as the adhesive for removing moisture (340), a material capable of removing moisture may be used.

According to an embodiment, referring to FIG. 4, when a sample is obtained using the microdialysis method, the microdialysis probe (300) may be fixed on a tissue using the mesh (330) and the adhesive (320 in the FIG. 4). For example, moisture exuded from a tissue may be present on the tissue (310 in the FIG. 4) from which the sample is to be obtained. In this case, when the microdialysis probe (300) is fixed on the tissue (310) using only the adhesive (320), the adhesive (320) may not be fixed on the tissue (310) due to the moisture exuded from the tissue. Here, the mesh (330) may absorb moisture exuded from the tissue (310). As the mesh (330) absorbs moisture, the microdialysis probe (300) may be firmly fixed on the tissue (310) by the adhesive (320).

According to another embodiment, referring to FIG. 4, when a sample is obtained using the microdialysis method, the microdialysis probe (300) may be fixed on the tissue (310) using the mesh (330), the adhesive (320) and the adhesive for removing moisture (340). Specifically, the mesh 330 may be adhered onto the tissue (310) to primarily absorb the moisture exuded from the tissue. In this case, the adhesive for removing moisture (340) can absorb the remaining moisture among the moisture exuded from the tissue (310), which the mesh (330) has primarily absorbed. Here, the adhesive (320) can fix the microdialysis probe (300) on the tissue from which the moisture has been removed.

Furthermore, an angle (a in the FIG. 4) at which the microdialysis probe (300) is inserted may be specified.

According to an embodiment, the microdialysis probe (300) may be inserted and fixed at an angle of 90°. For example, when a position into which the microdialysis probe (300) is inserted is the brain, the microdialysis probe (300) may be inserted at an angle of 90° in order to be inserted into a desired position on the brain tissue.

According to still embodiment, the microdialysis probe (300) may be inserted and fixed at an angle of 45°. For example, when a position into which the microdialysis probe (300) is inserted is the dorsal horn of the spinal cord, the microdialysis probe (300) may be inserted at an angle of 45° in order to be inserted into a desired position on the dorsal horn of the spinal cord.

However, the angle at which the microdialysis probe (300) is inserted is not limited to the above-described example, and the angle (a) at which the microdialysis probe (300) is inserted may vary depending on the type of tissue into which the microdialysis probe (300) is inserted.

Hereinafter, the analysis of neurotransmitters using a brain map will be described in detail with reference to some examples.

Analysis of neurotransmitters using brain map

2.1 Object of Analysis

The brain map may be used to measure the concentrations of neurotransmitters.

According to an embodiment, the brain map may be used in order to evaluate the efficacy of a drug. For example, the brain map may be used to measure changes in the concentrations of neurotransmitters in the brain after administration of a drug associated with a mental disorder. Here, when the change in the concentration of a neurotransmitter, which changes after the administration of the mental disorder-associated drug is large, it can be evaluated that the efficacy of the drug is good.

However, the drug to be administered in the present specification is not limited to the mental disorder-associated drug, and the drug to be administered may be a drug associated with diseases of parts of the body other than the brain or spinal cord.

According to another embodiment, the brain map can be used to measure the concentration of a specific neurotransmitter in the brain of a patient with a mental disorder. For example, in the case of a patient with depression, the brain map may be used to measure the patient's dopamine at a position where dopamine is present at high concentration.

According to another embodiment, the brain map may be used in order to predict a mental disorder. Specifically, when the concentration of a specific neurotransmitter at a specific position differs by a predetermined level or more compared to a brain map of a normal brain, the brain map may be used to predict the presence of a mental disorder due to an excess or lack of the specific neurotransmitter.

The purpose of measuring neurotransmitters using the brain map is not limited to the above-described examples, and the brain map can also be used for an additional purpose to confirm the concentrations of neurotransmitters in the brain. However, hereinafter, for the convenience of description, the brain map will be used to measure the concentration of a neurotransmitter in order to evaluate the efficacy of a drug.

2.2 Analysis Target Position

The brain map may be used to select a position from which a sample is obtained in order to analyze the neurotransmitter.

The position from which the sample is obtained may be a position into which the above-described microdialysis probe (300 in the FIG. 3 or FIG. 4) is inserted.

The specific neurotransmitter may be a neurotransmitter that the drug intends to change. For example, when a drug to treat depression is treated, the neurotransmitter which the drug intends to change may be dopamine.

The brain map may provide a position where the concentration of a specific neurotransmitter is highest.

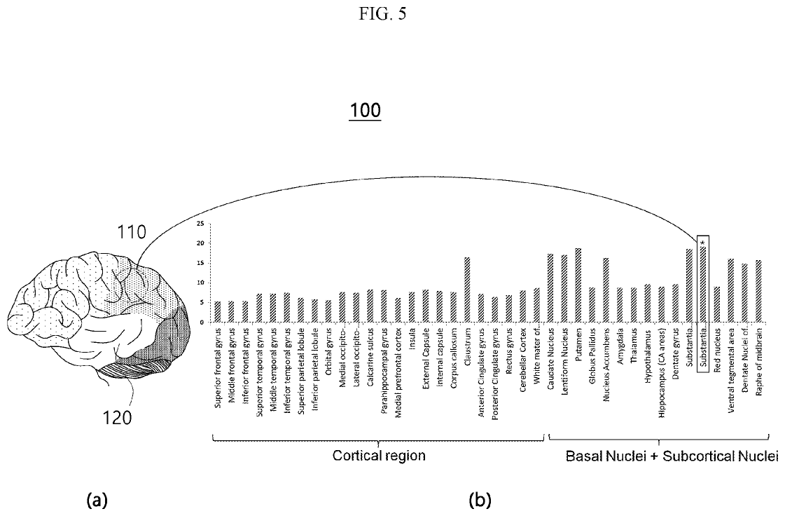

FIG. 5(a) is a schematic view illustrating a brain map in which the concentration of a neurotransmitter is illustrated at each position of the brain according to an embodiment.

FIG. 5(b) is a graph showing the concentration of a neurotransmitter (dopamine) at each position of the brain according to an embodiment.

The horizontal axis in FIG. 5(b) represents each region of the brain, and the vertical axis represents the concentration of a neurotransmitter.

Referring to FIG. 5(b), the specific transmitter may have different concentrations for each position of the brain. Accordingly, a position where the concentration of a specific neurotransmitter is highest may be selected.

The position where the concentration of a specific neurotransmitter is highest may be a position where a change in the concentration of a specific neurotransmitter can be clearly measured.

However, the position for measuring the specific neurotransmitter is not limited to the above-described position, and may be a position where the concentration of the specific neurotransmitter is a predetermined level or more.

According to an embodiment, the brain map can provide a position where the concentration of a neurotransmitter, which the drug intends to change, is highest. For example, when the neurotransmitter, which the drug intends to change, is dopamine, the region where the concentration of dopamine is highest in FIG. 5(b) may be a first region (110) in FIG. 5(a).

In this case, as the brain map according to an embodiment of the present invention, a brain map of a target animal other than a human may be used. For example, the brain map to be used may be a brain map of a rhesus monkey.

Alternatively, the brain map according to another embodiment of the present invention may be a brain map in which a human brain map matches a brain map of a target animal. Here, the matching of the human brain map with the brain map of the target animal may match a position where the concentration distribution of a neurotransmitter in the target animal brain and the concentration distribution of the neurotransmitter in the human brain are similar. The matching of the brain map of the target animal with the human brain map will be described in detail in the related parts below.

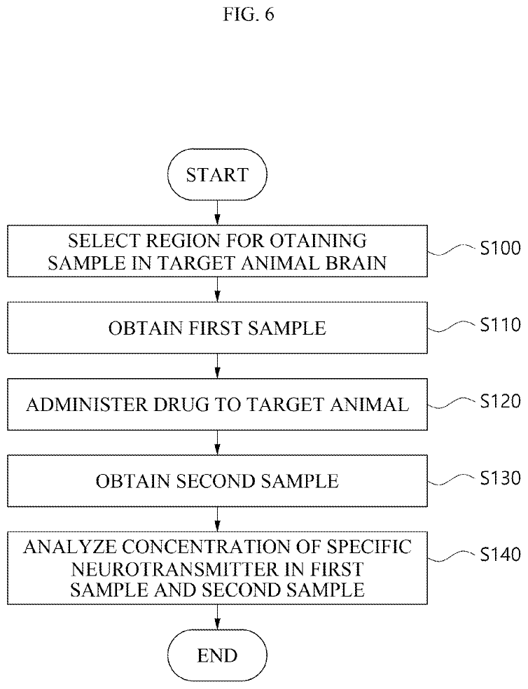

FIG. 6 is a flow chart for explaining a method for measuring the concentration of a neurotransmitter using a brain map in order to evaluate the efficacy of a drug in a target animal according to an embodiment.

Here, the neurotransmitter to be measured may be a neurotransmitter which a drug intends to change in order to evaluate the efficacy of the drug. The neurotransmitter to be measured may be one particular neurotransmitter or a plurality of neurotransmitters. For example, a neurotransmitter which a drug associated with the treatment of depression intends to change may be dopamine. Alternatively, a neurotransmitter which a drug associated with pain intends to change may be glutamate or GAB A.

Hereinafter, referring to FIG. 6, the measurement of a neurotransmitter using a brain map will be described with reference to some examples.

According to an embodiment, a region for obtaining a sample may be selected in a target animal brain (S100 in the FIG. 6). Here, the target animal may be a target animal from which a sample is obtained using the above-described microdialysis method.

As a more specific example, the brain map used to select the region for obtaining the sample may be a brain map indicating the concentrations of neurotransmitters in the target animal. Here, the region to be selected may be a position where the concentration of a specific neurotransmitter is highest in the brain map of the target animal.

According to another embodiment, the brain map used to select the region for obtaining the sample (S100) may be a brain map indicating the concentrations of neurotransmitters in the target animal. Here, the region to be selected may be a position where the concentration of a specific neurotransmitter is a predetermined level or more in the target animal brain.

Here, the predetermined level of the concentration of the specific neurotransmitter may be a level at which the degree of change of the specific neurotransmitter can be distinguished when the obtained sample is analyzed. A level at which the degree of change can be distinguished may mean that the detection sensitivity or detection accuracy of an analytical instrument is a predetermined level or more when the obtained sample is analyzed. For example, when the specific neurotransmitter is dopamine, the concentration of dopamine in the region for obtaining the sample may be 15 ng/ml or more.

According to still another embodiment, the brain map used to select the region for obtaining the sample (S100) may be a brain map in which a human brain map matches a brain map of a target animal to be a target from which a sample is obtained. Accordingly, the region to be selected may be a position in the brain map of the target animal, which matches a position where the concentration of the specific neurotransmitter in the human brain map is highest.

As a more specific example, the human brain map may be a brain map in which the concentrations of neurotransmitters are indicated for each position in the human brain. Further, the brain map of the target animal may be a brain map in which the concentrations of neurotransmitters are indicated for each position in the target animal brain. The brain map in which the human brain map matches the target animal brain may be a brain map in which the human brain map matches the target animal brain map based on the tendency of the concentrations of neurotransmitters.

According to yet another embodiment, as the brain map used to select a region for obtaining a sample (S100), it is possible to use a brain map of a model with a disease that has or is predicted to have a therapeutic effect depending on a drug to be administered (hereinafter, referred to as a disease model) and a brain map of a model without a disease (hereinafter, referred to as a normal model). As an example, the disease model may be a target animal with a disease that has or is predicted to have a therapeutic effect depending on a drug to be administered. In addition, the normal model may be a target animal without any disease. However, the disease model and the normal model are not limited to the target animal, and may be a human with a disease who has or is predicted to have a therapeutic effect depending on a drug to be administered and a human without any disease.

As a more specific example, the specific neurotransmitter may have different concentrations in the disease model compared to the normal model due to the presence of the disease. When the disease of the disease model suppresses the secretion of the specific neurotransmitter, the concentration of the specific neurotransmitter may be lower in the disease model than in the normal model. Alternatively, when the disease of the disease model promotes the secretion of the specific neurotransmitter, the concentration of the specific neurotransmitter may be higher in the disease model than in the normal model. Accordingly, the concentrations of the specific neurotransmitter in the disease model and the normal model may be different from each other.

The region for obtaining the sample may be selected as a position where the concentrations of a specific neurotransmitter differ by a predetermined level or more by comparing the brain map of the disease model with the brain map of the normal model. For example, the extent to which the concentrations of a specific neurotransmitter differ may be a 50% or more different from the concentration of the specific neurotransmitter in the normal model.

According to yet another embodiment, the brain map used to select the region for obtaining the sample (S100) may be a brain map in which the concentrations of a plurality of neurotransmitters are indicated.

The region for obtaining the sample may be a plurality of positions. Specifically, when the injected drug changes a plurality of neurotransmitters, the region for obtaining a sample can be selected as a position where the plurality of neurotransmitters have the highest concentration.

According to yet another embodiment, the brain map used to select the region for obtaining the sample (S100) may be a brain map in which the concentrations of a plurality of neurotransmitters are indicated in an overlapping manner.

As a more specific example, the position for obtaining the sample may be selected as a position where the positions where the concentrations of a plurality of neurotransmitters are high are overlapping. Specifically, when the injected drug changes a plurality of neurotransmitters, there may be a plurality of changed neurotransmitters. In this case, positions where the concentrations of a plurality of neurotransmitters are a predetermined level or more may overlap. Overlapping positions may be one position or a plurality of positions, but may be less than the number of positions where each of the concentrations of a plurality of neurotransmitters is highest, so that when a sample is obtained using a microdialysis method, a position into which a microdialysis probe is inserted may be minimized.

Here, the predetermined level of the concentrations of the plurality of neurotransmitters for selecting the overlapping position may be a level at which the degree of change of the plurality of neurotransmitters can be distinguished when the obtained sample is analyzed. A level at which the degree of change can be distinguished may mean that the detection sensitivity or detection accuracy of an analytical instrument is a predetermined level or more when the obtained sample is analyzed. For example, when one neurotransmitter of the plurality of neurotransmitters to be analyzed is dopamine, the concentration of dopamine for selecting the overlapped region may be 15 ng/ml or more.

Referring to FIG. 6, a first sample may be obtained based on the region selected using the brain map (S110). Here, the first sample may be a sample obtained using the above-described microdialysis method. The first sample may have the highest concentration of a neurotransmitter which the drug intends to change compared to samples obtained from other positions in the brain. Referring to FIG. 6, a drug may be administered to a target animal (S120).

The drug to be administered may change the concentration of a specific neurotransmitter. The drug to be administered may be a drug associated with a mental disorder, including donepezil, venlafaxine and Aricept. The drug to be administered may be a drug which has an analgesic component to suppress pain. However, the drug is not limited thereto, and all drugs which change neurotransmitters in the brain may be included among the drugs which may be administered.

Referring to FIG. 6, a second sample may be obtained from the region where the first sample is obtained (S130). The second sample may be obtained after a certain period of time has passed after the drug was administered. The second sample may be a sample obtained using the above-described microdialysis method, like the first sample.

Referring to FIG. 6, the concentration of the specific neurotransmitter in the first sample and the second sample may be analyzed (S140). The concentration of the specific neurotransmitter may be analyzed using a device capable of analyzing the mass of fine molecules, such as an LC-MS/MS, a GC-MS, an electron capture detector (ECD) and an Elisa device.

2.3 Method of Evaluating Efficacy of Drug Using Brain Map

The brain map may be used to evaluate the efficacy of a drug associated with a disease.

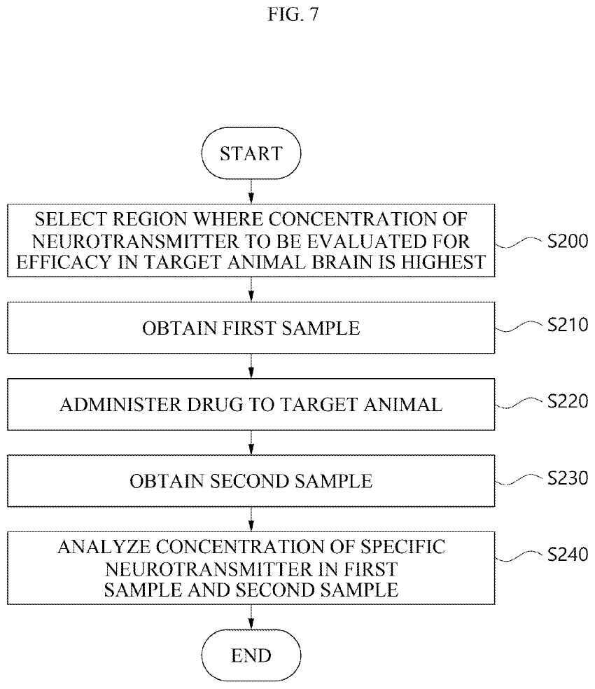

FIG. 7 is a flow chart for explaining a method for measuring the concentration of a neurotransmitter in a region of a target animal brain where the concentration of a neurotransmitter is highest, using a brain map in order to evaluate the efficacy of a drug in the target animal according to an embodiment.

Referring to FIG. 7, the selecting of the region where the concentration of a neurotransmitter to be evaluated for efficacy in the target animal brain is highest (S200) may include selecting the above-described position to be analyzed as a region where the concentration of the neurotransmitter is highest. Specifically, the region for obtaining the sample may be a region where the concentration of a neurotransmitter which is a target to be evaluated for efficacy using the brain map is highest. For example, when the neurotransmitter which a specific drug changes is dopamine, the region for obtaining the sample may be a region where the concentration of dopamine is highest in the brain map of the target animal.

Referring to FIG. 7, the obtaining of the first sample (S210) and the obtaining of the second sample (S230) may include obtaining a sample using the above-described method of obtaining a sample for analysis. For example, the obtaining of the sample may include obtaining a sample using the microdialysis method.

Referring to FIG. 7, the administration of the drug into the target animal (S220) may include injecting the drug into the body of the target animal such that the drug has an effect. Specifically, the drug may be orally administered to an animal to be subjected to an experiment. Here, the method of orally administering the drug is not limited to a method in which the animal to be subjected to an experiment autonomously ingests the drug, and includes a method of inserting a catheter into the esophagus and forcibly administering the drug through the catheter. Alternatively, the drug may be intraperitoneally administered into an animal to be subjected to an experiment. Here, the method of intraperitoneally administering the drug may be a method of injecting a drug into a digestive organ such as the stomach or intestines. Alternatively, the method of administering a drug into the abdominal cavity may be a method of inserting a catheter into a digestive organ through the oral cavity, and then injecting a drug by the catheter. Alternatively, the drug may be intravenously administered into an animal to be subjected to an experiment through injection. Specifically, the drug to be administered may be in a liquid form. Accordingly, the drug may be intravenously administered into the animal to be subjected to an experiment using a syringe. However, the method of administering the drug is not limited to the above-described example, and all known drug administration methods, such as subcutaneous injection of the drug into the animal to be subjected to an experiment or administration of the drug into the animal to be subjected to an experiment through the ductus arteriosus, may be the drug administration method according to the present specification.

Referring to FIG. 7, the analyzing of the concentration of the specific neurotransmitter in the first sample and the second sample (S240) may include analyzing the above-described neurotransmitter. As a specific example, for the concentration of the specific neurotransmitter, the concentration of the specific neurotransmitter in the first sample and the second sample may be analyzed using a device capable of analyzing the mass of fine molecules, such as an LC-MS/MS, a GC-MS, an electron capture detector (ECD) and an Elisa device.

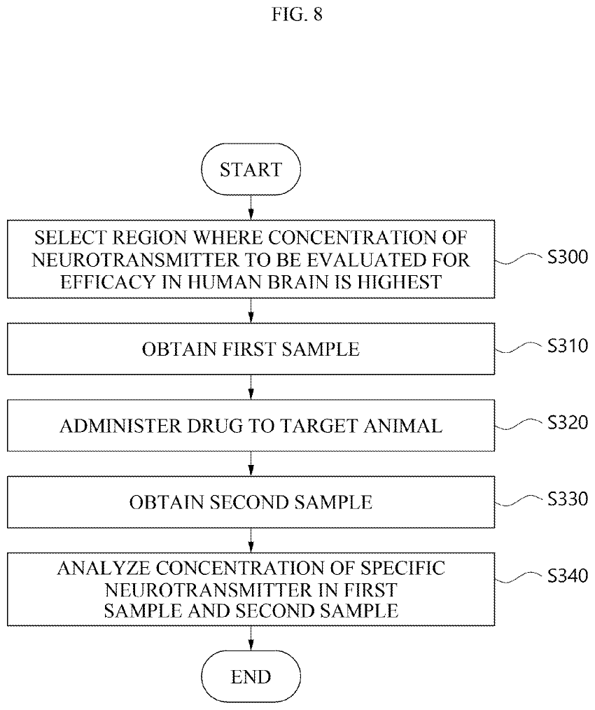

FIG. 8 is a flow chart for explaining a method for measuring the concentration of a neurotransmitter in a region of a target animal brain, which matches a region where the concentration of a neurotransmitter to be evaluated for drug efficacy in the human brain is highest, using a brain map in order to evaluate the efficacy of a drug in the target animal according to an embodiment.

Referring to FIG. 8, the selecting of the region in the target animal brain, which matches the region where the concentration of a neurotransmitter to be evaluated for efficacy in the human brain is highest (S300) may include selecting the above-described position to be analyzed as a region where the concentration of a neurotransmitter is highest. Specifically, the region for obtaining the sample may be a position on the brain map of the target animal, which matches a region where the concentration of a neurotransmitter, which the drug intends to change in the human brain map is highest. For example, when the neurotransmitter, which the specific drug changes, is dopamine, the region for obtaining the sample may be a position of the target animal brain, which matches a position where dopamine in the human brain map is present at the highest concentration.

Referring to FIG. 8, the obtaining of the first sample (S310) and the obtaining of the second sample (S330) may include obtaining a sample using the above-described method of obtaining a sample for analysis. For example, the obtaining of the sample may include obtaining a sample using the microdialysis method.

Referring to FIG. 8, the administering of the drug into the target animal (S320) may include injecting the drug into the body of the target animal such that the drug has an effect. Specifically, the drug may be orally administered to an animal to be subjected to an experiment. Here, the method of orally administering the drug is not limited to a method in which the animal to be subjected to an experiment autonomously ingests the drug, and includes a method of inserting a catheter into the esophagus and forcibly administering the drug through the catheter. Alternatively, the drug may be intraperitoneally administered into an animal to be subjected to an experiment. Here, the method of intraperitoneally administering the drug may be a method of injecting a drug into a digestive organ such as the stomach or intestines. Alternatively, the method of administering a drug into the abdominal cavity may be a method of inserting a catheter into a digestive organ through the oral cavity, and then injecting a drug by the catheter. Alternatively, the drug may be intravenously administered into an animal to be subjected to an experiment through injection. Specifically, the drug to be administered may be in a liquid form. Accordingly, the drug may be intravenously administered into the animal to be subjected to an experiment using a syringe. However, the method of administering the drug is not limited to the above-described example, and all known drug administration methods, such as subcutaneous injection of the drug into the animal to be subjected to an experiment or administration of the drug into the animal to be subjected to an experiment through the ductus arteriosus, may be the drug administration method according to the present specification.

Referring to FIG. 8, the analyzing of the concentration of the specific neurotransmitter in the first sample and the second sample (S240) may include analyzing the above-described neurotransmitter. As a specific example, for the concentration of the specific neurotransmitter, the concentration of the specific neurotransmitter in the first sample and the second sample may be analyzed using a device capable of analyzing the mass of fine molecules, such as an LC-MS/MS, a GC-MS, an electron capture detector (ECD) and an Elisa device.

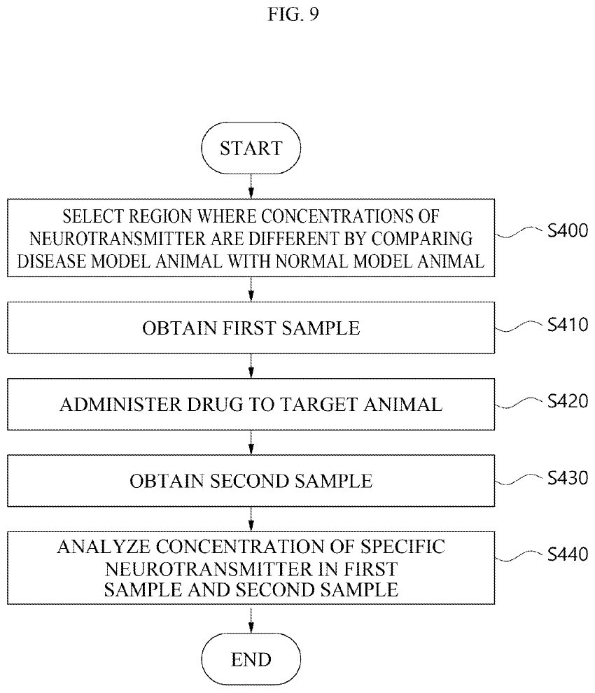

FIG. 9 is a flow chart for explaining a method for measuring the concentration of a neurotransmitter in regions where the concentrations of the neurotransmitter are different by comparing a disease model with a normal model in a target animal, using a brain map in order to evaluate the efficacy of a drug in the target animal according to an embodiment.

Referring to FIG. 9, the selecting of the region where the concentration of a neurotransmitter to be evaluated for efficacy in the target animal brain is highest (S400) may include selecting the above-described position to be analyzed as a region where the concentrations of the neurotransmitter in the disease model and the normal model are different. Specifically, the region for obtaining the sample may be a region where the concentrations of the neurotransmitter are different using a brain map of a disease model and a brain map of a normal model among target animals. For example, a neurotransmitter which has different concentrations of the neurotransmitter in the brain map of the disease model animal and the brain map of the normal model animal may be dopamine, and regions where dopamine is present at different concentrations in the brain maps of the disease model and the normal model may also be selected.

Referring to FIG. 9, the obtaining of the first sample (S410) and the obtaining of the second sample (S430) may include obtaining a sample using the above-described method of obtaining a sample for analysis. For example, the obtaining of the sample may include obtaining a sample using the microdialysis method.

Referring to FIG. 9, the administering of the drug into the target animal (S420) may include injecting the drug into the body of the target animal such that the drug has an effect. Specifically, the drug may be orally administered to an animal to be subjected to an experiment. Here, the method of orally administering the drug is not limited to a method in which the animal to be subjected to an experiment autonomously ingests the drug, and includes a method of inserting a catheter into the esophagus and forcibly administering the drug through the catheter. Alternatively, the drug may be intraperitoneally administered into an animal to be subjected to an experiment. Here, the method of intraperitoneally administering the drug may be a method of injecting a drug into a digestive organ such as the stomach or intestines. Alternatively, the method of administering a drug into the abdominal cavity may be a method of inserting a catheter into a digestive organ through the oral cavity, and then injecting a drug by the catheter. Alternatively, the drug may be intravenously administered into an animal to be subjected to an experiment through injection. Specifically, the drug to be administered may be in a liquid form. Accordingly, the drug may be intravenously administered into the animal to be subjected to an experiment using a syringe. However, the method of administering the drug is not limited to the above-described example, and all known drug administration methods, such as subcutaneous injection of the drug into the animal to be subjected to an experiment or administration of the drug into the animal to be subjected to an experiment through the ductus arteriosus, may be the drug administration method according to the present specification.

Referring to FIG. 9, the analyzing of the concentration of the specific neurotransmitter in the first sample and the second sample (S440) may include analyzing the above-described neurotransmitter. As a specific example, for the concentration of the specific neurotransmitter, the concentration of the specific neurotransmitter in the first sample and the second sample may be analyzed using a device capable of analyzing the mass of fine molecules, such as an LC-MS/MS, a GC-MS, an electron capture detector (ECD) and an Elisa device.

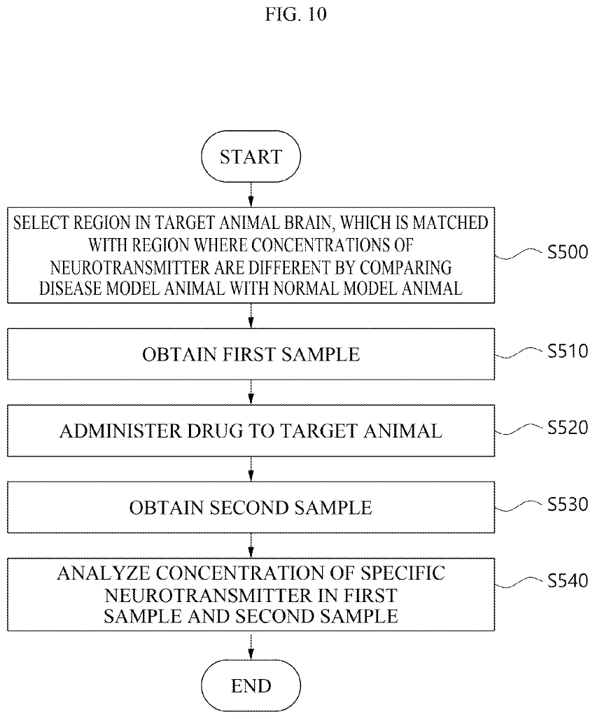

FIG. 10 is a flow chart for explaining a method for measuring the concentration of a neurotransmitter in a region in a target animal brain, which matches regions where the concentrations of the neurotransmitter are different by comparing a disease model human with a normal model human, using a brain map in order to evaluate the efficacy of a drug in the target animal according to an embodiment.

Referring to FIG. 10, the selecting of the region in the target animal brain, which matches the region where the concentrations of the neurotransmitter are different by comparing the disease model human with the normal model human (S500) may include selecting the above-described position to be analyzed as a region in the target animal brain, which matches a region where the concentrations of the neurotransmitter are different in the human disease model and the human normal model. Specifically, for the region for obtaining the sample, a region where the concentrations of the neurotransmitter are different using a brain map of a disease model and a brain map of a normal model may be a matched region in the brain map of the target animal. For example, a neurotransmitter which has different concentrations of neurotransmitters in the brain map of the human disease model and the brain map of the human normal model may be dopamine, and a region in the target animal brain, which matches the region where dopamine is present at different concentrations in the brain maps of the human disease model and the human normal model, may also be selected.

Referring to FIG. 10, the obtaining of the first sample (S510) and the obtaining of the second sample (S530) may include obtaining a sample using the above-described method of obtaining a sample for analysis. For example, the obtaining of the sample may include obtaining a sample using the microdialysis method.

Referring to FIG. 10, the administering of the drug into the target animal (S520) may include injecting the drug into the body of the target animal such that the drug has an effect. Specifically, the drug may be orally administered to an animal to be subjected to an experiment. Here, the method of orally administering the drug is not limited to a method in which the animal to be subjected to an experiment autonomously take the drug, and includes a method of inserting a catheter into the esophagus and forcibly administering the drug through the catheter. Alternatively, the drug may be intraperitoneally administered into an animal to be subjected to an experiment. Here, the method of intraperitoneally administering the drug may be a method of injecting a drug into a digestive organ such as the stomach or intestines. Alternatively, the method of administering a drug into the abdominal cavity may be a method of inserting a catheter into a digestive organ through the oral cavity, and then injecting a drug by the catheter. Alternatively, the drug may be intravenously administered into an animal to be subjected to an experiment through injection. Specifically, the drug to be administered may be in a liquid form. Accordingly, the drug may be intravenously administered into the animal to be subjected to an experiment using a syringe. However, the method of administering the drug is not limited to the above-described example, and all known drug administration methods, such as subcutaneous injection of the drug into the animal to be subjected to an experiment or administration of the drug into the animal to be subjected to an experiment through the ductus arteriosus, may be the drug administration method according to the present specification.

Referring to FIG. 10, the analyzing of the concentration of the specific neurotransmitter in the first sample and the second sample (S540) may include analyzing the above-described neurotransmitter. As a specific example, for the concentration of the specific neurotransmitter, the concentration of the specific neurotransmitter in the first sample and the second sample may be analyzed using a device capable of analyzing the mass of fine molecules, such as an LC-MS/MS, a GC-MS, an electron capture detector (ECD) and an Elisa device.

The analysis method of neurotransmitters using the brain map disclosed by the present specification is not limited to the examples described above, and may be implemented in any form including a method of analyzing neurotransmitters by selecting a region where a neurotransmitter is analyzed through a brain map which indicates the neurotransmitter and an index associated with the neurotransmitter, and using a sample obtained from the selected position.

Hereinafter, a method for make a brain map used in order to analyze a neurotransmitter will be described.

3 Make of Brain Map

A brain map for make and analyzing the concentration of a neurotransmitter in a specific region can be produced.

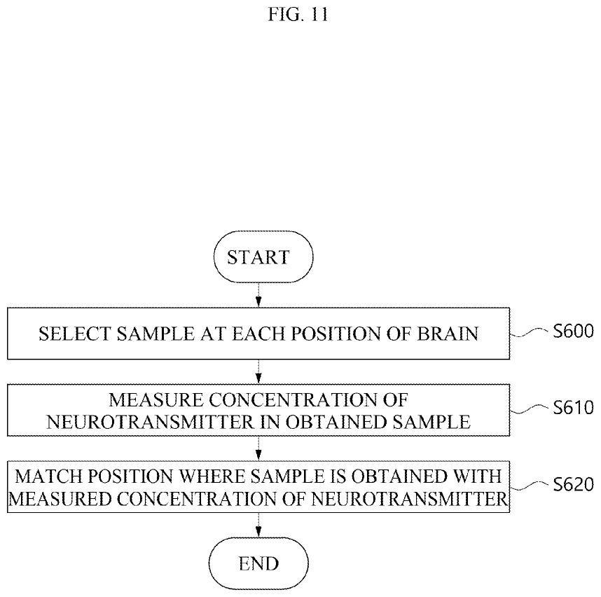

FIG. 11 is a flow chart for showing that a brain map is made based on neurotransmitters according to an embodiment.

Referring to FIG. 11, the make of the brain map may include obtaining a sample at each position of the brain (S600). The make of the brain map may include measuring the concentration of a neurotransmitter in a sample obtained at each position of the brain (S610). The make of the brain map may include matching the measured concentration of the neurotransmitter with the position where the sample is obtained (S620).

In the obtaining of the sample for each position of the brain (S600), a method of obtaining a sample for measuring the concentration of neurotransmitters may be included.

As a more specific example, the obtaining of the sample for each position of the brain (S600) may include obtaining a sample using a microdialysis method in an in-vivo state. Alternatively, the obtaining of the sample for each position of the sample (S600) may include obtaining a sample for each position of the brain by removing the brain of a target animal. Alternatively, the obtaining of the sample for each position of the brain (S600) may include obtaining a sample by performing a tissue biopsy on each position of the target animal brain in an in-vivo state. However, the step is not limited to the above-described examples, and various methods capable of obtaining a sample at each position of the brain may be included in the obtaining of the sample for each position of the brain. The obtaining of the sample for each position of the brain (S600) will be described in more detail in the method of obtaining a sample for make a brain map.

In the measuring of the concentration of the neurotransmitter in the obtained sample (S610), a measurement method for measuring the concentration of the neurotransmitter may be included.

As a more specific example, the measuring of the concentration of the neurotransmitter in the obtained sample (S610) may include measuring the concentration of the neurotransmitter using a mass spectrometer. However, the measuring of the concentration of the neurotransmitter in the obtained sample (S610) is not limited thereto, and a brain map may be made by measuring the concentration of a neurotransmitter receptor, the protein expression level according to the metabolism of the neurotransmitter and the concentration of mRNA. Hereinafter, for convenience of description, the measuring of the concentration of the neurotransmitter in the obtained sample (S610) is described as including measuring the concentration of the neurotransmitter in the obtained sample. The measuring of the concentration of the neurotransmitter in the obtained sample (S610) will be hereinafter described in more detail in the method for measuring the neurotransmitter.

The matching of the measured concentration of neurotransmitters with the position where the sample is obtained (S620) may include matching a position of a sample obtained at each position of the brain of the target animal with the concentration of the neurotransmitter measured in the obtained sample. The matching of the measured concentration of the neurotransmitter with the position where the sample is obtained (S620) will be hereinafter described in more detail in the matching of each position of the brain with the concentration of the neurotransmitter.

Hereinafter, each step for make a brain map will be described with reference to some examples.

3.1 Method of Obtaining Sample for Make Brain Map

A brain map may be made based on a sample obtained at each position of the brain of an animal including a human.

Hereinafter, a method of obtaining a sample will be described with reference to some examples.

3.1.1 Obtaining of Sample Using Microdialysis Method

According to an embodiment, a sample to be obtained may be a sample obtained using a microdialysis method in an in-vivo state. For example, the microdialysis probe 300 (in the FIG. 3 or FIG. 4) may be inserted into each position of the brain of a target animal. In this case, the sample to be obtained may be a sample in which a position where the sample is obtained is indicated.

Specifically, the position into which the microdialysis probe (300) is inserted may be a position which is anatomically responsible for a specific function. For example, the microdialysis probe (300) may be inserted into a region which is responsible for different functions, such as the thalamus, medulla, and cerebral cortex. In the inserted microdialysis probe (300), a sample may be obtained by exchanging an artificial cerebrospinal fluid (aCSF) with a tissue fluid at the insertion position.

In this case, the microdialysis probe (300) may be fixed at the insertion position by the above-described microdialysis fixture method.

3.1.2 Obtaining of Sample by Removing Brain of Target Animal

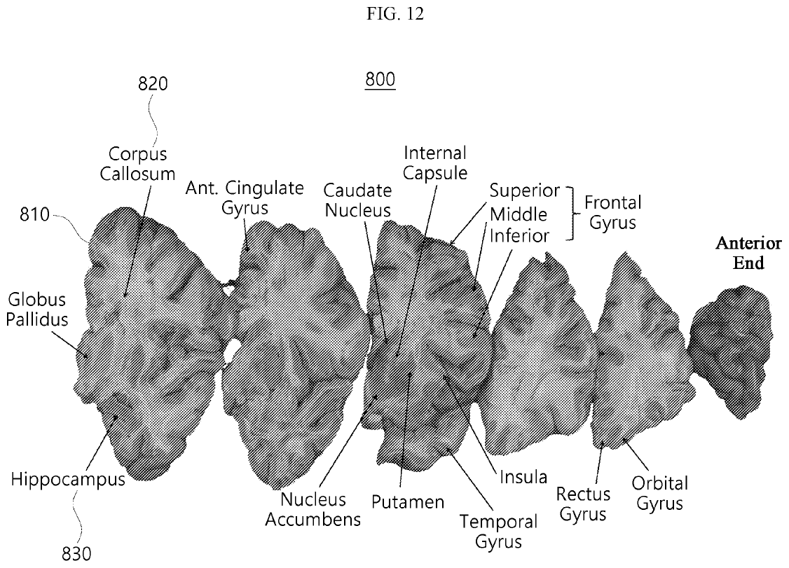

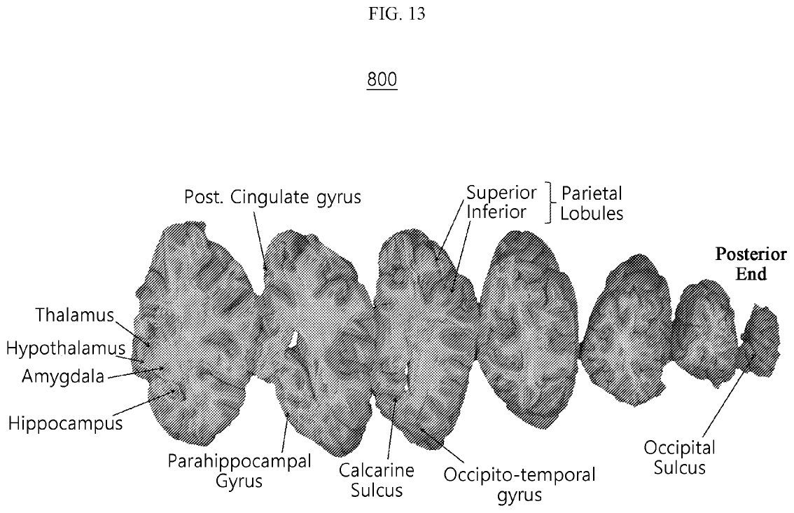

FIGS. 12 and 13 are view showing that the human brain is cut into even sizes according to an embodiment.

Referring to FIGS. 12 and 13, a sample to be obtained may be obtained from the brain cut to a uniform thickness after removing the brain of a target animal. In this case, the position corresponding to the brain before being cut may be indicated on the brain cut to an even thickness.

Hereinafter, for convenience of description, the brain cut to an even thickness will be referred to as a brain section.

Referring to FIGS. 12 and 13, a sample to be obtained may be obtained using a microdialysis method on brain sections (800 in the FIG. 12 or FIG. 13). Specifically, a first sample-obtaining position (820 in the FIG. 12) and a second sample-obtaining position (830 in the FIG. 12) may be present on a first brain section (810 in the FIG. 12). Here, the first sample-obtaining position (820) and the second sample-obtaining position (830) may be positions which are responsible for different functions in the brain. Accordingly, different neurotransmitter concentration distributions may appear in samples obtained from the first sample-obtaining position (820) and the second sample-obtaining position (830).

However, a position where a sample is obtained is not limited to the first sample-obtaining position (820) and the second sample-obtaining position (830), and a sample can be obtained even in another region of the brain. In addition, the first sample-obtaining position (820) and the second sample-obtaining position (830) may not be regions which are responsible for a specific function in the brain, and may be any region on the brain.

Hereinafter, some examples will be given to describe the obtaining of the sample from the removed brain of a target animal.

3.1.2.1 Obtaining of Sample from Brain Section Through Microdialysis Method

Referring to FIG. 12, a sample to be obtained may be obtained from the first sample-obtaining position (820) and the second sample-obtaining position (830) using the above-described microdialysis method.

As a more specific example, the microdialysis probe (300 in the FIG. 3 or FIG. 4) may be inserted into the first sample-obtaining position (820) and the second sample-obtaining position (830). In the inserted microdialysis probe (300), a sample may be obtained by exchanging an artificial cerebrospinal fluid with a tissue fluid at the insertion position.

In this case, on a sample to be obtained, the first sample-obtaining position (820) and the second sample-obtaining position (830) may be indicated.

3.1.2.2 Obtaining of Sample by Punching Brain Section

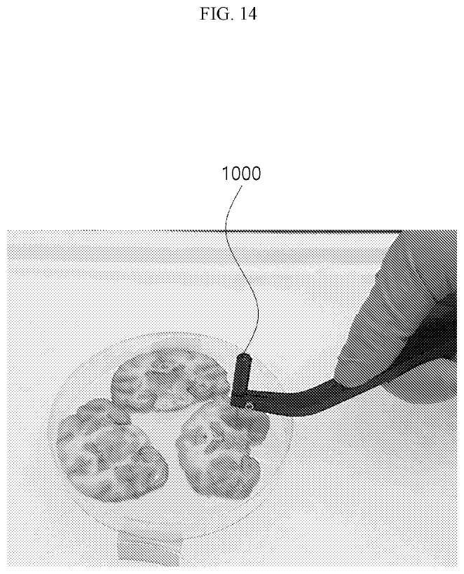

FIG. 14 is a view showing that a sample is obtained by punching a section of the brain according to an embodiment. Here, punching means a method of making a hole in a predetermined portion and removing the corresponding portion. In this case, punching may be performed by a punch (1000). The punch (1000) means a device that has a predetermined size and can make a hole in an object.

Referring to FIGS. 12 and 13, a sample to be obtained may be obtained from the first sample-obtaining position (820) and the second sample-obtaining position (830) using the above-described punching.

As a more specific example, the punch (1000) may perform punching at the first sample-obtaining position (820) and the second sample-obtaining position (830). In this case, the punched and separated tissue may be a sample.

As an example, the punched and separated tissue may be ground and used as a sample. Here, the punched and separated tissue may be dissolved and crushed in a solution such as an artificial cerebrospinal fluid, physiological saline and/or water. Furthermore, the sample to be obtained may be a supernatant obtained after the tissue dissolved and crushed in the solution is separated into a solid and a solution using a centrifuge.

However, the method of obtaining a sample at each position of the brain section is not limited to punching, and may include both of a method of obtaining a sample by cutting the sectioned brain to a unit size and a method capable of separating a tissue at each position of the brain of the target animal.

In this case, on a sample to be obtained, the first sample-obtaining position (820) and the second sample-obtaining position (830) may be indicated.

3.1.3 Method of Obtaining Additional Sample

A sample to be obtained may be obtained by biopsy at each position of the brain of a target animal. Here, the biopsy may be performed by any means of separating tissue from each position of the brain.

As an example, the biopsy may be performed at each position of the brain using biopsy forceps. Specifically, a sample to be obtained may be obtained by separating the tissue using biopsy forceps. The tissue separated using biopsy forceps may be separated using a centrifuge after being dissolved and crushed in a solution as in the method of obtaining a sample using the above-described punching method. Accordingly, the sample to be obtained may be a supernatant of a solution separated using a centrifuge.

As another example, the biopsy may be performed at each position of the brain using biopsy forceps. Specifically, the sample to be obtained may be obtained by separating tissue at each position of the brain of the target animal using a syringe. The tissue separated using a syringe may be separated using a centrifuge after being dissolved and crushed in a solution as in the method of obtaining a sample using the above-described punching method. Accordingly, the sample to be obtained may be a supernatant of a solution separated using a centrifuge.

Here, the method of obtaining a sample is not limited to those described above, and all methods capable of obtaining a sample including a neurotransmitter for each region of the brain of a target animal may be included in the method of obtaining a sample.

3.2 Method for Measuring Neurotransmitter

The concentration of a neurotransmitter may be obtained from the obtained sample.

According to an embodiment, the concentration of a neurotransmitter of the sample to be obtained may be measured using a mass spectrometer.

As an example, the mass spectrometer may be a liquid chromatography-tandem mass spectrometer (LC-MS/MS). Specifically, in the sample to be obtained, the concentration of a neurotransmitter may be measured using a LC-MS/MS device. Here, for the sample to be obtained, neurotransmitters may be separated by concentration by a liquid chromatography method. The concentration of each separated neurotransmitter may be measured using a mass spectrometer.

As another example, the mass spectrometer may be a gas chromatography-mass spectrometer (GC-MS/MS). Specifically, in the sample to be obtained, the concentration of a neurotransmitter may be measured using a GC-MS/MS device. Here, for the sample to be obtained, neurotransmitters may be separated by concentration by a gas chromatography method. The concentration of each separated neurotransmitter may be measured using a mass spectrometer.

As still another example, the mass spectrometer may be an electron capture detector (ECD). Specifically, in the sample to be obtained, the concentration of a neurotransmitter may be measured using an ECD device. The sample to be obtained may include a liquid chromatography step or a gas chromatography step as a pretreatment step before analysis by a BCD device.

As yet another embodiment, the concentration of a neurotransmitter of the sample to be obtained may be measured by an enzyme-linked immunosorbent assay (ELISA) method. Specifically, in the case of a neurotransmitter based on the protein in the sample to be obtained, the concentration may be measured using an antigen-antibody reaction in the ELISA.

However, the method for measuring the concentration of a neurotransmitter in the obtained sample is not limited to the above-described example, and all methods for measuring the concentration of a neurotransmitter using the obtained sample may be included.

3.3 Matching of Concentration of Neurotransmitter with Each Position of Brain

Referring to the above-described FIG. 5, the measured concentration of a neurotransmitter may be matched with the position where the sample is obtained.

According to an embodiment, the matching of each position of the brain with the concentration of a neurotransmitter may include matching using the measured concentration of the neurotransmitter and the position where the sample is obtained as described above.

As a more specific example, referring to FIG. 5B, the concentration of a specific neurotransmitter may be known by measuring the obtained sample. In this case, when the obtained sample is matched with an existing position, the concentration of the neurotransmitter at a position where the sample is obtained may be matched.

Further, referring to FIG. 5(a), the measured concentration of a neurotransmitter and the position may be shown on a schematic view (hereinafter, referred to as a brain map) of the brain.

As an example, each position where the concentration of the brain map of FIG. 5(a) is indicated may indicate the concentration distribution of a specific neurotransmitter. That is, FIG. 5(a) may be a brain map which indicates the concentration distribution of a specific neurotransmitter. Here, each of the divided regions in FIG. 5(a) may be a region expressed from a high concentration to a low concentration of the specific neurotransmitter.

As another example, the first region (110) and the second region (120) of the brain map of FIG. 5(a) may indicate different neurotransmitters. Specifically, the first region (110) may indicate the highest concentration of a neurotransmitter in the first region. Further, a second region (120) may indicate the highest concentration of a neurotransmitter in the second region.

3.4 Matching of Human Brain Map with Brain Map of Target Animal

According to an embodiment, a brain map in which a human brain map matches a brain map of the target animal may be made. Here, the matching of the human brain map with the brain map of the target animal may be used to perform an experiment by searching for the corresponding position in the target animal based on a position where a neurotransmitter is specified in the human brain map.

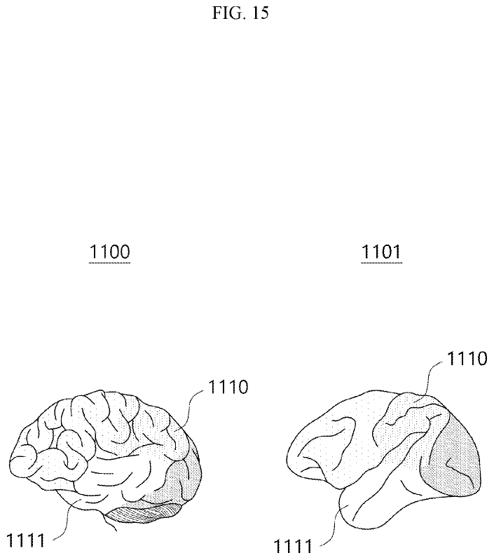

FIG. 15 is a schematic view showing that that a human brain map and a monkey brain map according to an embodiment are matched based on the similarity in neurotransmitter concentrations.

Referring to FIG. 15, the human brain map (1100) may be matched with the brain map of the target animal (1101). Specifically, a first corresponding region (1110) may be a position where the concentration distribution of a first neurotransmitter shows a similar distribution between the human brain map (1100) and the brain map of the target animal (1101). In addition, a second corresponding region (1111) may be a position where the concentration distribution of a second neurotransmitter shows a similar distribution between the human brain map (1100) and the brain map of the target animal (1101).

For example, the first corresponding region (1110) may be a position indicating where the concentration of dopamine is distributed on the human brain map with the highest concentration of dopamine, and similarly, may be a position indicating where the concentration of dopamine is also distributed on the brain map of the target animal with the highest concentration of dopamine. Furthermore, the second corresponding region (1111) may be a position indicating where the concentration of GABA is distributed on the human brain map with the highest concentration of GABA, and similarly, may be a position indicating where the concentration of dopamine is also distributed on the brain map of the target animal with the highest concentration of dopamine.

However, the human brain map and the brain map of the target animal do not correspond only to the first corresponding region (1110) and the second corresponding region (1111), and a position where a similar neurotransmitter concentration distribution is shown may be the corresponding region where both the human brain map and the brain map of the target animal are matched.

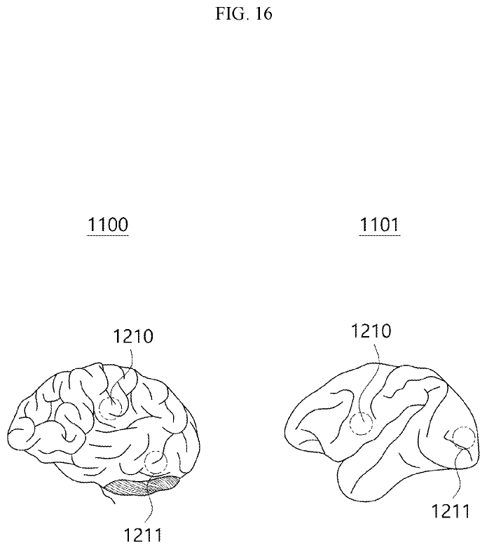

Further, FIG. 16 is a view illustrating the anatomically identical positions of the human brain and the monkey brain according to an embodiment. Here, the target animal may be a monkey.

Referring to FIG. 16, the human brain and the brain of the target animal may have positions which have the same anatomical function. That is, the human brain map and the animal brain map may be matched with positions having the same anatomical function.

As an example, a first identical functional region (1210) may be a region which processes cognitive functions associated with hearing, and a second identical functional region (1211) may be a region which processes cognitive functions associated with vision. In this case, the processing functions may be the same in the first identical functional region (1210), but the concentrations of a neurotransmitter may be different in the human brain and the brain of the target animal. Likewise, the processing functions may be also the same in the second identical functional region (1211), but the concentrations of a neurotransmitter may be different in the human brain and the target animal brain.

Accordingly, the matching of the human brain map with the brain map of the target animal may match the positions which perform the same function for each position of the brain.

However, the human brain map and the brain map of the target animal do not correspond only to the first identical functional region (1210) and the second identical functional region (1211), and a position having a similar function may be the corresponding region where both the human brain map and the brain map of the target animal are matched.

The matching of the human brain map with the brain map of the target animal is not limited to the above-described examples and may be based on a similarity such as the concentration of mRNA, the concentration of protein, and the concentration of a neurotransmitter receptor.

4 Examples for Selecting Neurotransmitter Measurement Region

A sample may be obtained by performing microdialysis on a region where a neurotransmitter to be analyzed is present using the concentration distribution of the neurotransmitter.

Hereinafter, a method of selecting a sample obtaining position for analysis of a specific neurotransmitter will be described in more detail.

4.1 Distribution of Neurotransmitters in Primate Brain

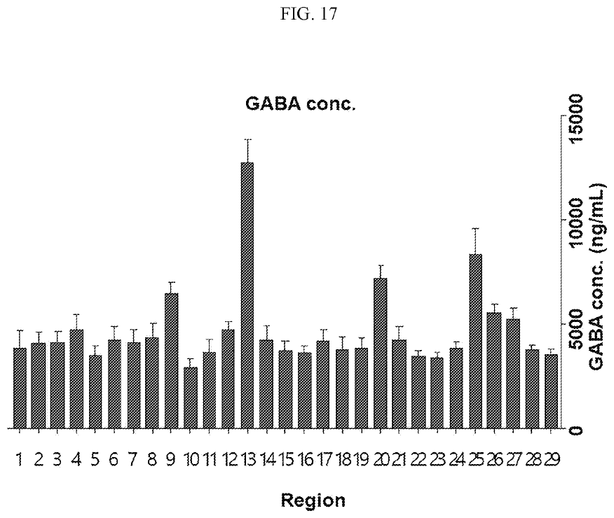

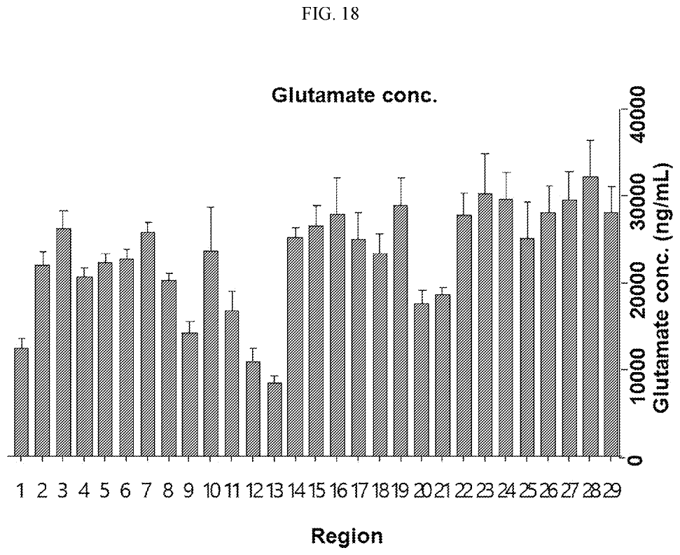

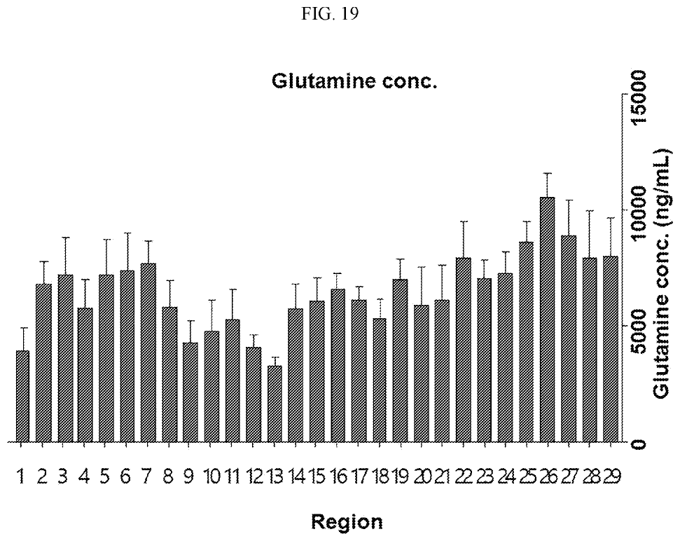

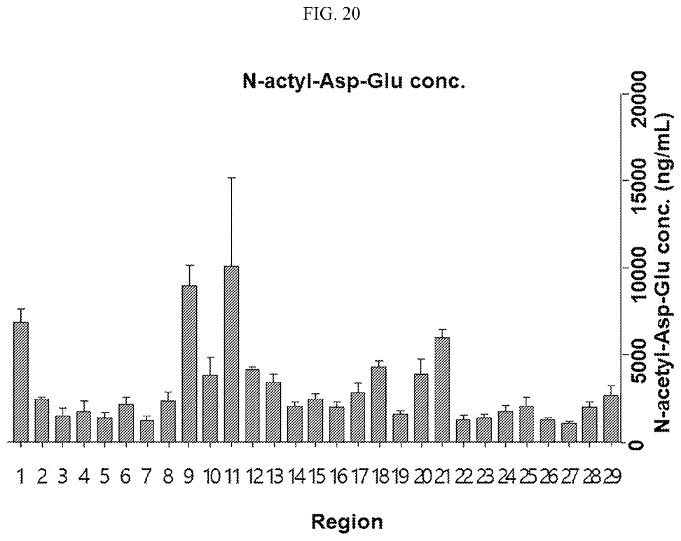

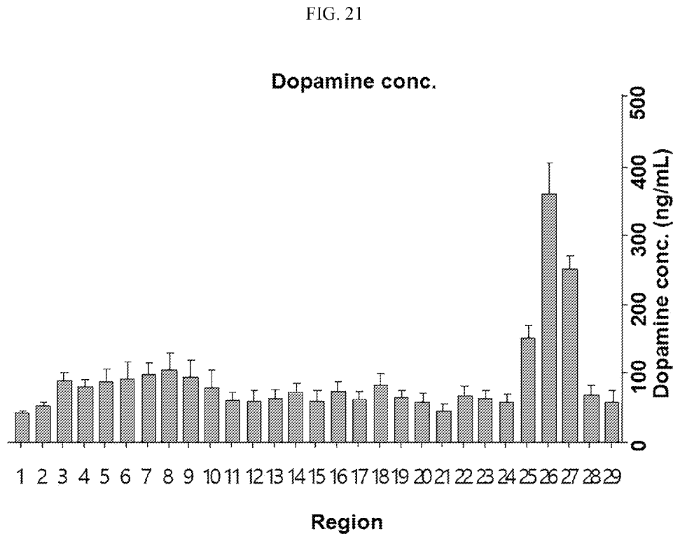

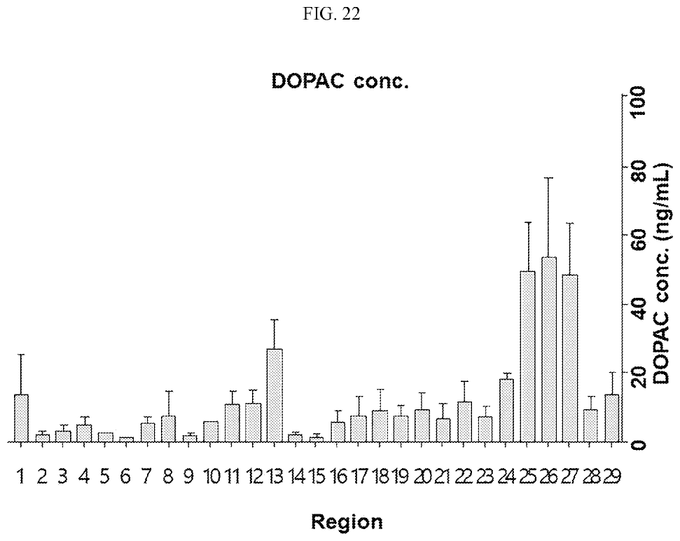

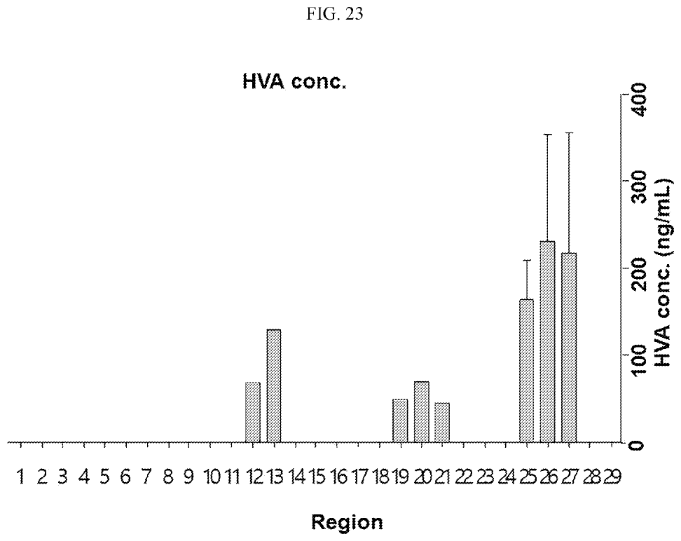

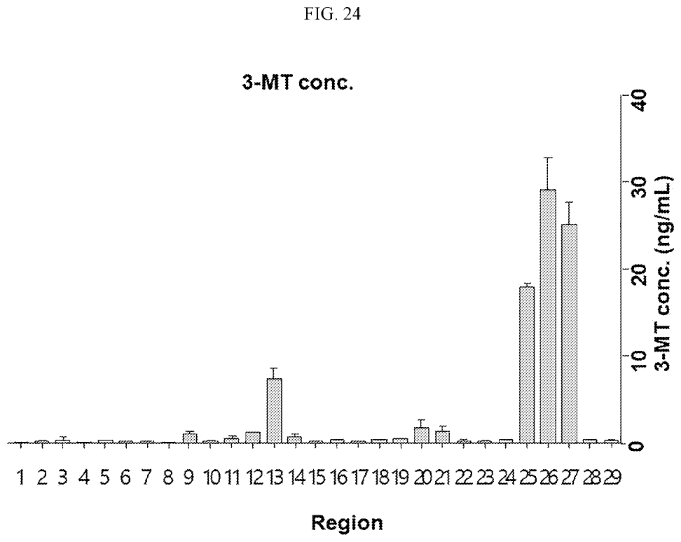

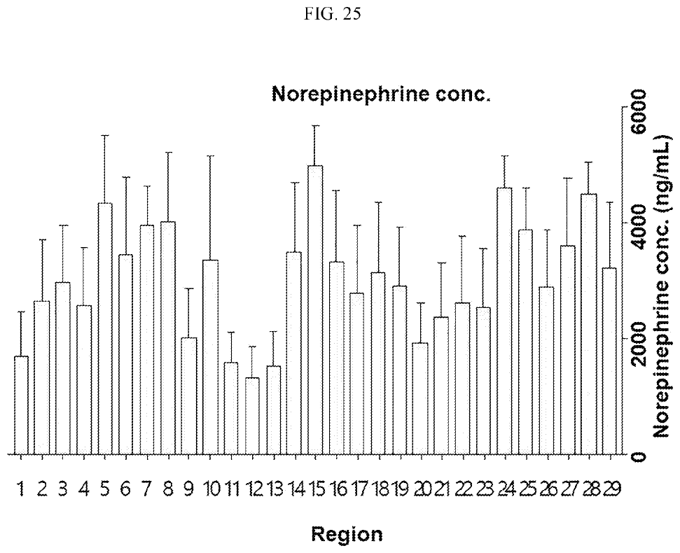

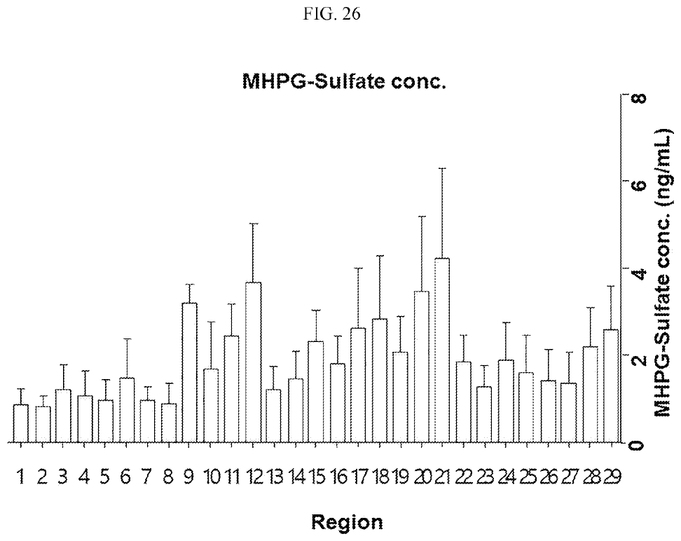

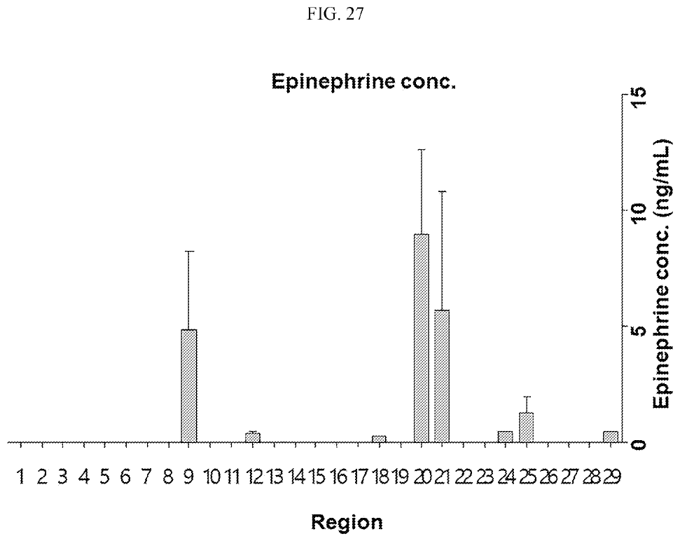

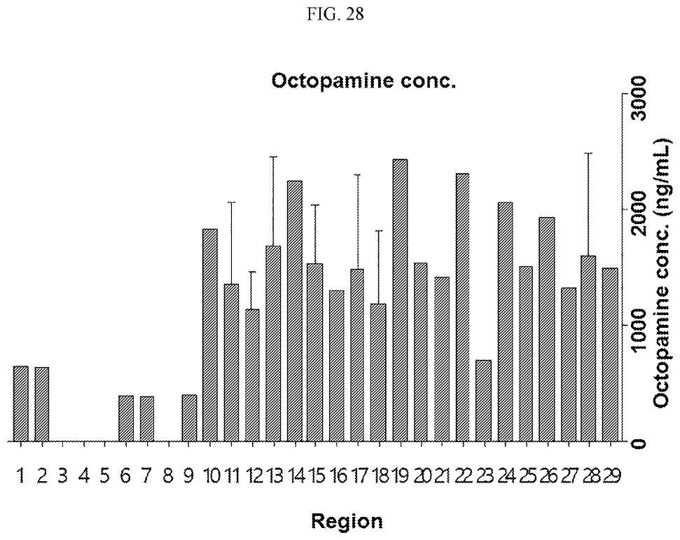

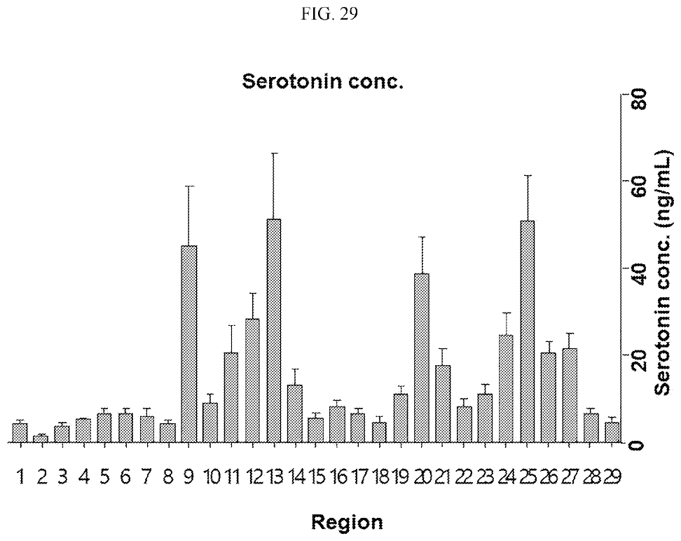

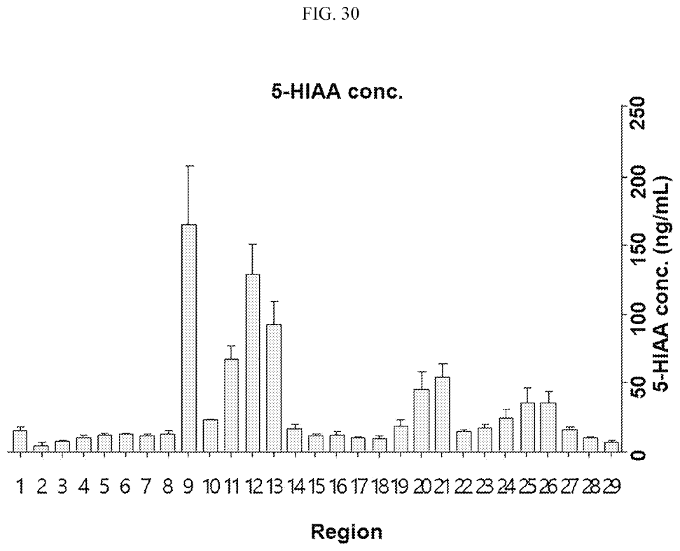

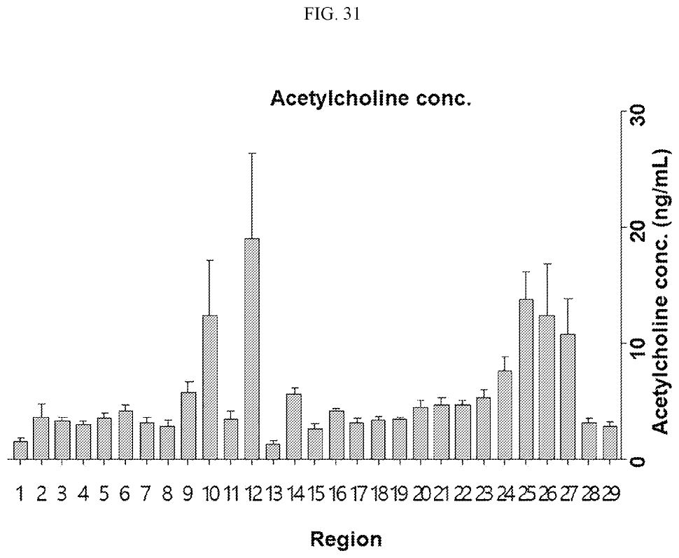

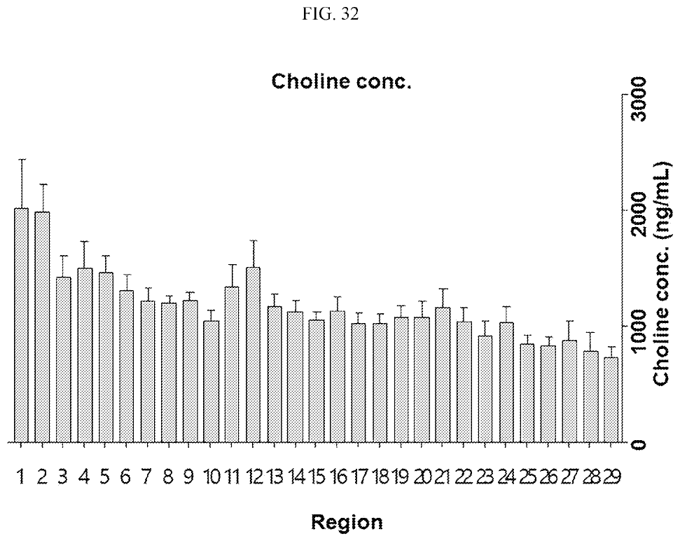

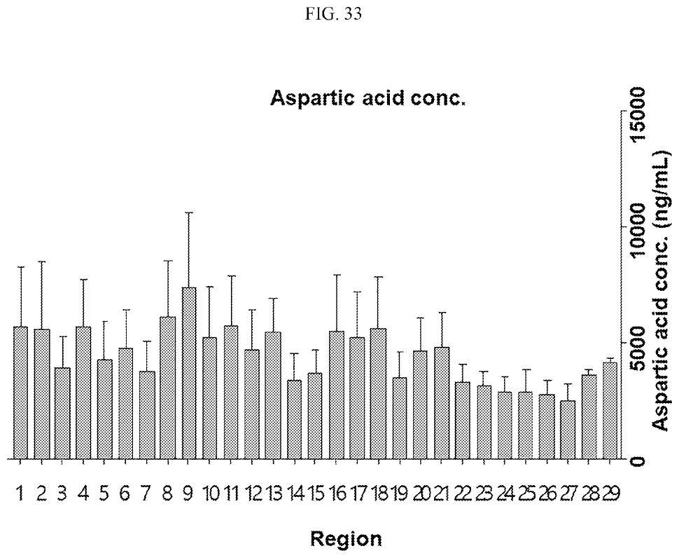

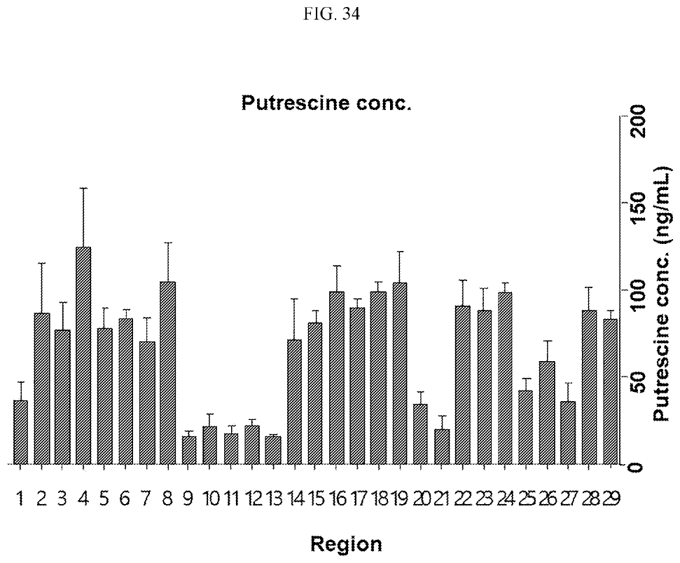

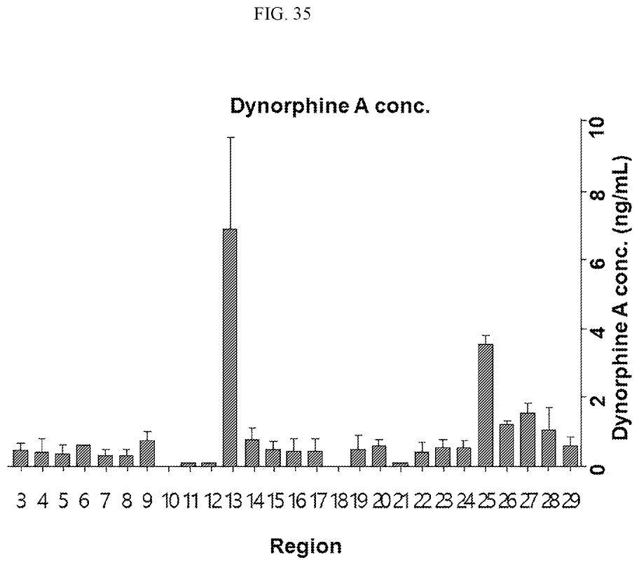

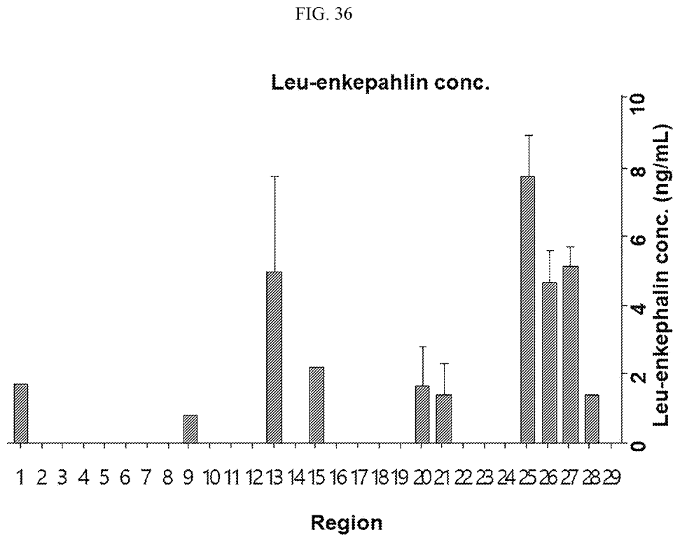

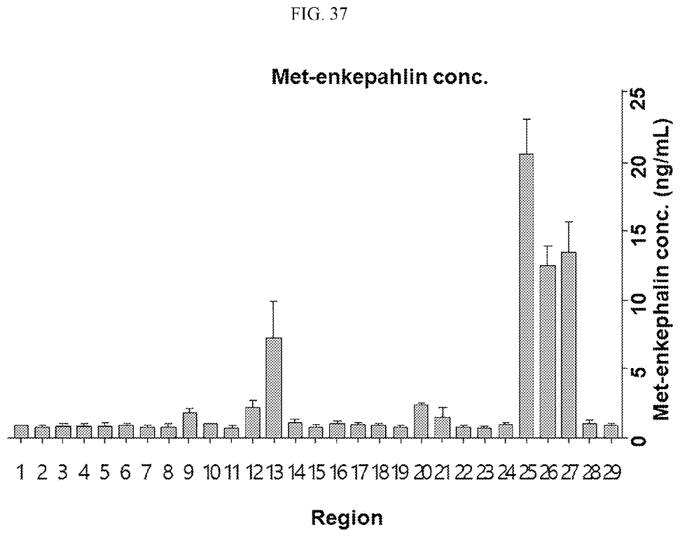

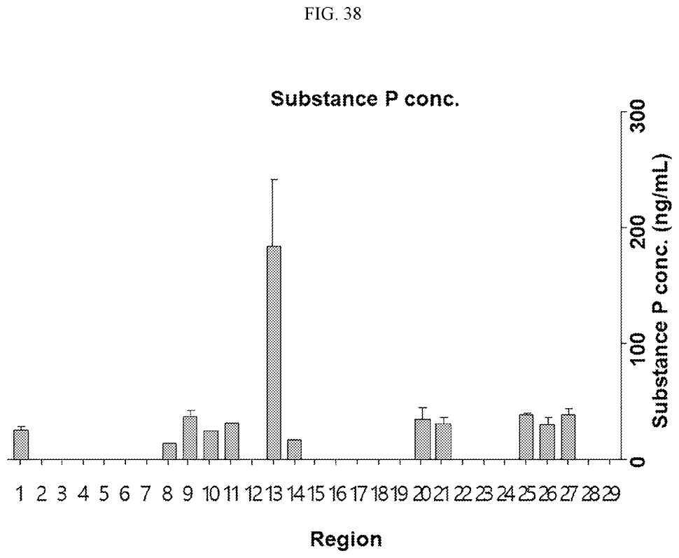

FIGS. 17 to 38 illustrate the concentration of each neurotransmitter at a plurality of positions in a primate brain according to an embodiment.

The horizontal axis of the graphs illustrated in FIGS. 17 to 38 represents each region of the primate brain, and the vertical axis represents a concentration at which each material is present in each region of the primate brain.

The following table shows a plurality of positions of the primate brain shown by the horizontal axis.

TABLE 1

| NO. | REGION NAME |
|---|---|
| 1 | Cerebellar Cortex-White Mater |
| 2 | Cerebellar Cortex-Gray Mater |
| 3 | Frontal Cortex |
| 4 | Occipital Cortex |
| 5 | Temporal Cortex |
| 6 | Parietal Cortex |
| 7 | Orbital Cortex |
| 8 | Visual Cortex |
| 9 | Superior Colliculus |
| 10 | Lateral Geniculate Body |
| 11 | Medial Geniculate Body |
| 12 | VTA |
| 13 | Substantia Nigra |
| 14 | Hippocampus |
| 15 | Posterior Cingulate Cortex |
| 16 | Auditory Cortex |
| 17 | Somatosensory Cortex |
| 18 | Motor Cortex |
| 19 | Insula |
| 20 | Hypothalamus |
| 21 | Thalamus |
| 22 | Perirhinal Cortex |
| 23 | Entorhinal Cortex |
| 24 | Periamygdaloid cortex |
| 25 | Nucleus Accumbens |
| 26 | Putamen |
| 27 | Caudate Nucleus |
| 28 | Anterior Cingulate Cortex |
| 29 | Medial PFc |

Each identification number associated with the plurality of regions shown on the horizontal axis of FIGS. 17 to 38 represents each region in the brain shown in Table 1 above.

4.2 Selection of Sample-Obtaining Region for Analysis of One Neurotransmitter

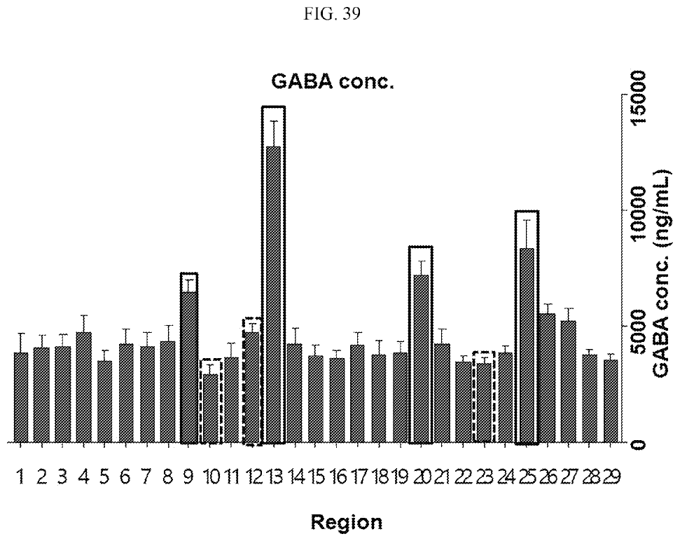

FIG. 39 is a graph showing the regions where samples can be obtained from the primate brain using the concentration distribution of GABA according to an embodiment.

According to an embodiment, a region where a specific neurotransmitter is present at a high concentration may be selected as a sample-obtaining position for analyzing the specific neurotransmitter.

According to another embodiment, a region where a specific neurotransmitter is present at a low concentration may not be selected as a sample-obtaining position for analyzing the specific neurotransmitter.

For example, referring to FIG. 39, a region where a sample for analyzing GABA, which is a specific neurotransmitter, is obtained, a region where GABA is distributed at a high concentration, and the regions of Superior colliculus, Substantia Nigra, Hypothalamus and Nucleus Accumbens indicated by solid lines may be selected. In addition, a region where GABA is distributed at a low concentration and the sample may not be obtained is a region represented by the dotted line, and the regions of Lateral Geniculate Body, VTA and Entorhinal Cortex may be selected.

However, the present invention is not limited thereto, and for a region where a sample for analyzing a specific neurotransmitter is obtained, a sample can be obtained from a region where the concentration of the specific neurotransmitter is a predetermined level or more. Further, a region which is not appropriate for obtaining a sample for analyzing a specific neurotransmitter may be a region where the concentration of the specific neurotransmitter is less than the predetermined level.

That is, when a single neurotransmitter is analyzed, a region where the concentration of the neurotransmitter to be analyzed is a predetermined level or more is selected as a region for obtaining the sample in order to improve the accuracy of the analysis.

4.3 Selection of Sample-Obtaining Region for Analyzing a Plurality of Neurotransmitters in One Region

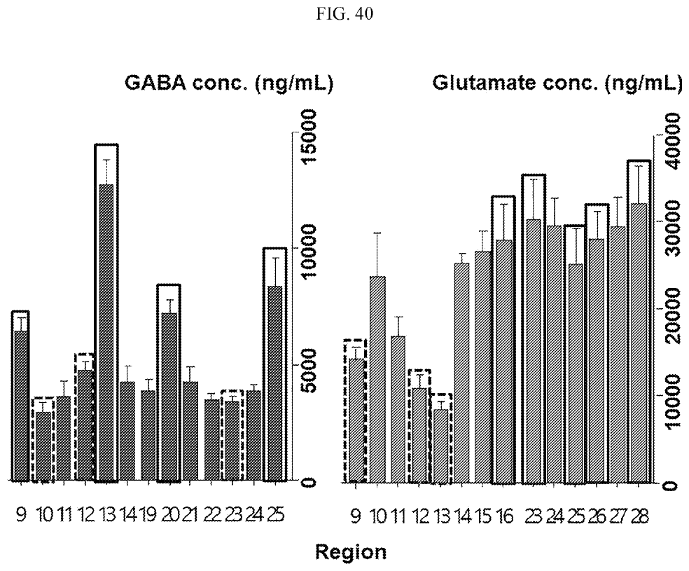

FIG. 40 is a graph showing the regions where samples are obtained for simultaneously analyzing GABA and glutamate in the primate brain according to an embodiment.

According to an embodiment, in order to minimize a position into which a microdialysis probe is inserted, a region where the concentrations of a plurality of neurotransmitters are present at a predetermined level or more may be selected as a position for obtaining a sample for simultaneously analyzing the plurality of neurotransmitters.

According to another embodiment, when the concentration of some neurotransmitters at a specific region is a predetermined level or more among a plurality of neurotransmitters, but the concentration of the remaining neurotransmitters is less than the predetermined level, the corresponding region may not be selected as a position where a sample for simultaneously analyzing a plurality of neurotransmitters is obtained.

According to still another embodiment, a region where the concentrations of a plurality of neurotransmitters are all less than a predetermined level may not be selected as a position where a sample for simultaneously analyzing the plurality of neurotransmitters is obtained.

For example, referring to FIG. 40, a region in the brain where the concentration of GABA among the plurality of neurotransmitters is a predetermined level or more may be Superior colliculus, Substantia Nigra, Hypothalamus and Nucleus Accumbens, which are represented by solid lines, and a region in the brain where the concentration of glutamate among the plurality of neurotransmitters is a predetermined level or more may be Auditory Cortex, Entorhinal Cortex, Nucleus Accumbens, Putamen and Anterior Cingulate Cortex, which are represented by solid lines. In addition, a region in the brain where the concentration of GABA is less than the predetermined level may be Lateral Geniculate Body, VTA and Entorhinal Cortex, which are represented by dotted lines, and a region in the brain where the concentration of glutamate is less than the predetermined level may be Superior Colliculus, VTA and Substantia Nigra, which are represented by dotted lines.

Here, as a region where a sample is obtained in order to simultaneously analyze GABA and glutamate, it is possible to select Nucleus Accumbens where the concentrations of GABA and glutamate are each predetermined levels or more. Furthermore, a region, which is not appropriate as a region where a sample is obtained in order to simultaneously analyze GABA and glutamate may be Substantia Nigra, VTA, Superior Colliculus and Entorhinal Cortex. In this case, the VTA region may not be appropriate as a position for obtaining a sample because the concentrations of GABA and glutamate are both less than predetermined levels. Further, in the Substantia Nigra and Superior Colliculus regions, the concentration of GABA is a predetermined level or more, but the concentration of glutamate is less than a predetermined level, so that the Substantia Nigra and Superior Colliculus regions may not be appropriate as regions for obtaining a sample. Likewise, in the Entorhinal Cortex region, the concentration of glutamate is a predetermined level or more, but the concentration of GABA is less than a predetermined level, so that the Entorhinal Cortex region may not be appropriate as a region for obtaining a sample.

However, the present invention is not limited thereto, and even though the concentrations of some neurotransmitters among the concentrations of a plurality of neurotransmitters in a specific region are less than a predetermined level, when the specific region is a region to which a microdialysis probe is easily accessible, the corresponding specific region may be used as a position for obtaining a sample for analyzing the concentrations of a plurality of neurotransmitters.

4.4 Selection of Sample-Obtaining Region for Analyzing Plurality of Neurotransmitters in Plurality of Regions

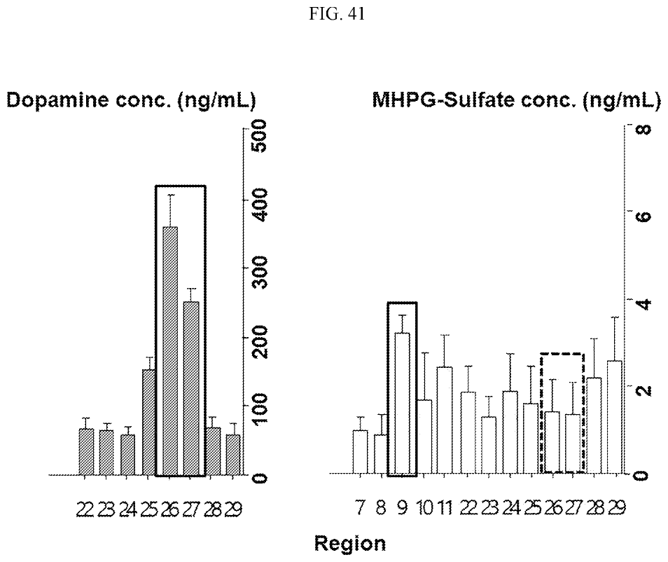

FIG. 41 is a graph showing the regions where samples are obtained for simultaneous analyzing dopamine and MHPG-sulfate from the primate brain according to an embodiment.

According to an embodiment, in order to minimize a position into which the means for obtaining a sample including a microdialysis probe is inserted, a plurality of regions in the brain may be selected as a region for obtaining a sample for simultaneously analyzing the concentrations of a plurality of neurotransmitters.

For example, referring to FIGS. 40 and 41 previously mentioned, a region in the brain where the concentration of dopamine among the plurality of neurotransmitters is a predetermined level or more may be Putamen and Caudate Nucleus, which are represented by solid lines, a region in the brain where the concentration of MHPG-sulfate among the plurality of neurotransmitters is a predetermined level or more may be Superior Colliculus, which is represented by a solid line, and a region where the concentration of MHPG-sulfate is less than the predetermined level may be Putamen and Caudate Nucleus, which are represented by dotted lines.

Here, since a region where the concentrations of previously mentioned GABA and glutamate are each a predetermined level or more does not coincide with a region where the concentrations of dopamine and MHPG-sulfate are each a predetermined level or more, samples may be respectively obtained in a plurality of regions.

More specifically, since the Putamen and Caudate Nucleus regions where the concentration of dopamine is a predetermined level or more are regions where the concentration of MHPG-sulfate is less than a predetermined level, the region may not be appropriate as a region for simultaneously analyzing dopamine and MHPG-sulfate. In this case, since the Superior Colliculus region has concentrations of both MHPG-sulfate and GABA, which are a predetermined level or more and the Putamen region has concentrations of both dopamine and glutamate that are a predetermined level or more, the number of positions into which the means for obtaining a sample is inserted may be minimized when selecting a region which overlaps with a region where the concentration of each neurotransmitter is a predetermined level or more. Accordingly, a sample for analyzing the concentrations of MHPG-sulfate and GABA may be obtained from the Superior Colliculus region, and a sample for analyzing the concentrations of dopamine and glutamate may be obtained from the Putamen region.

That is, in order to minimize a plurality of positions where the means for obtaining a sample, such as a microdialysis probe, is inserted into the brain, the region for obtaining the sample may be a region where regions where the concentrations of some neurotransmitters among the plurality of neurotransmitters are a predetermined level or more are overlapped with each other.

However, the present invention is not limited thereto, and even though the concentrations of some neurotransmitters among the concentrations of a plurality of neurotransmitters in a specific region are less than a predetermined level, when the specific region is a region to which a microdialysis probe is easily accessible, the corresponding specific region may be used as a position for obtaining a sample for analyzing the concentrations of a plurality of neurotransmitters.

5 Examples for Matching the Human Brain with the Primate Brain

5.1 Distribution of Neurotransmitters in Human Brain

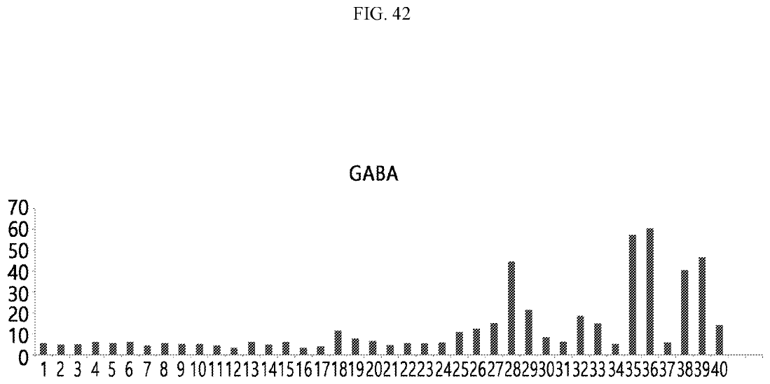

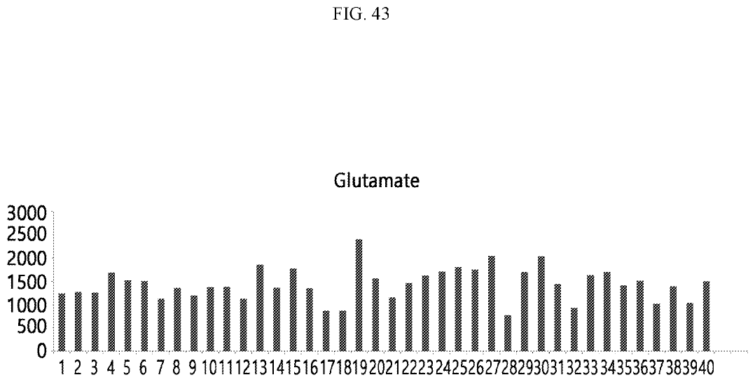

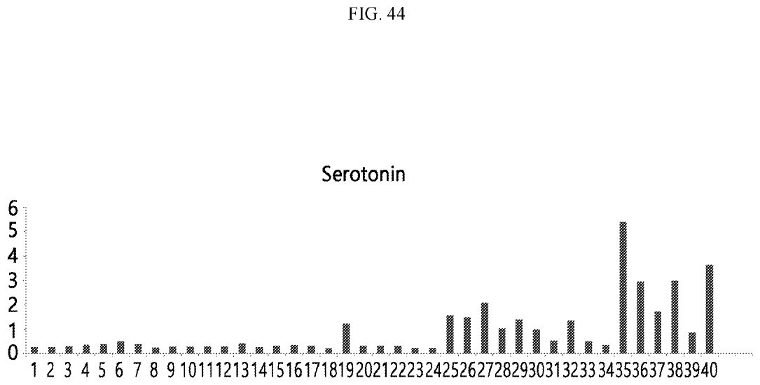

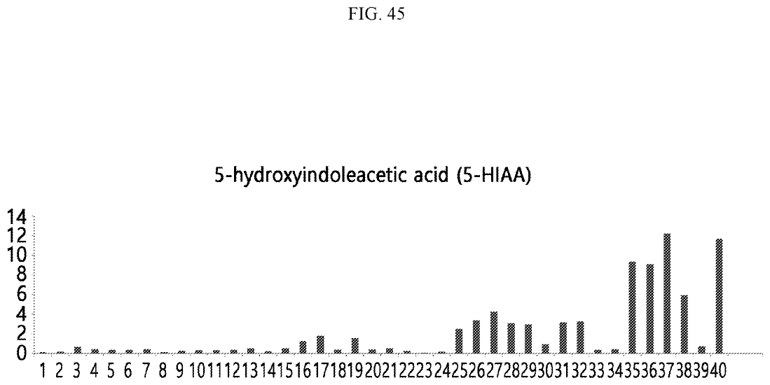

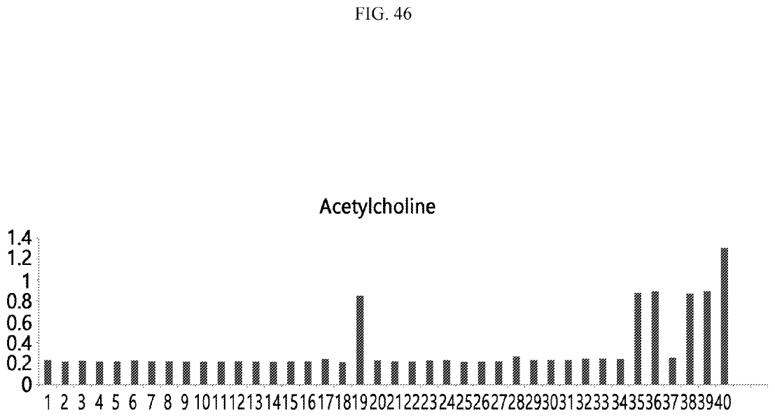

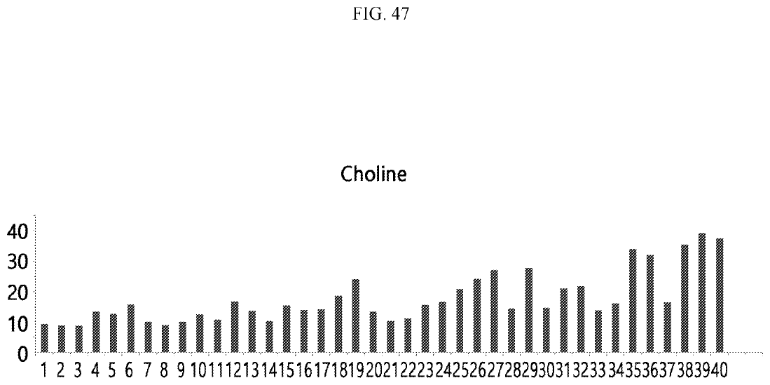

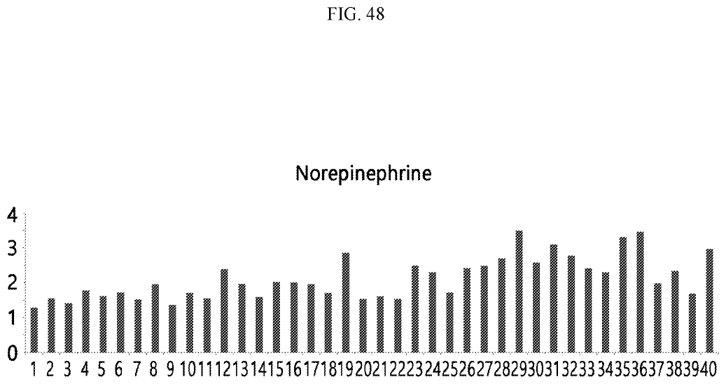

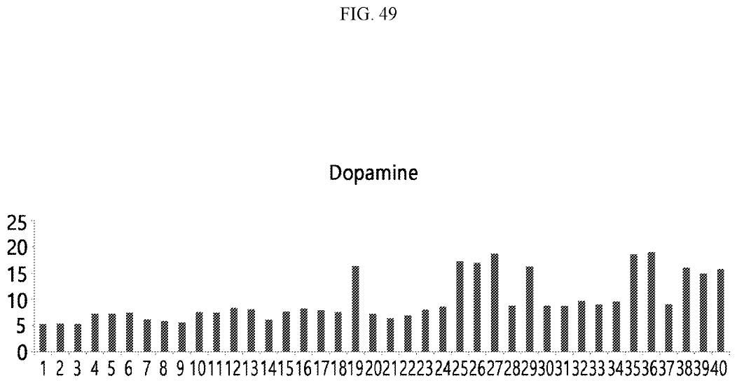

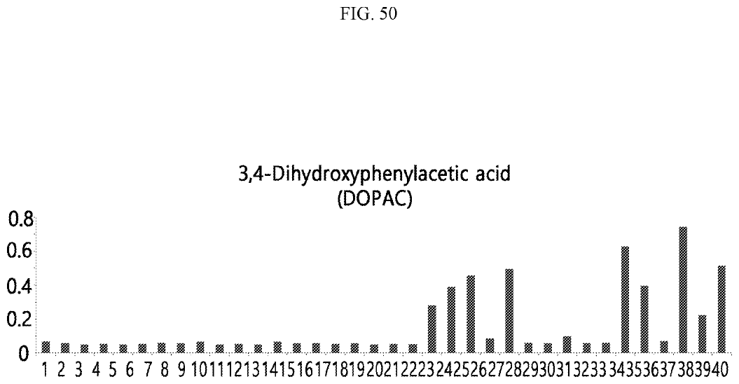

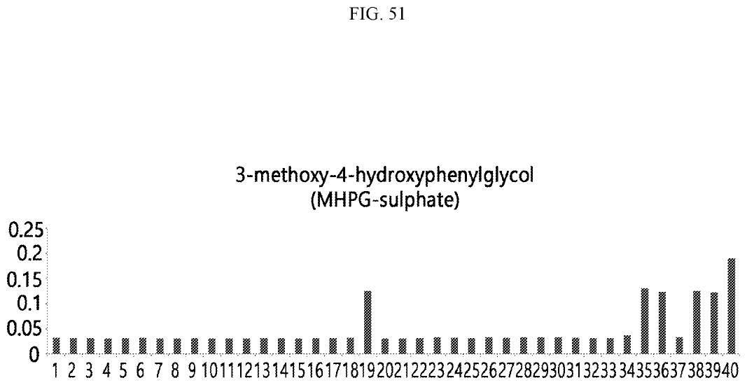

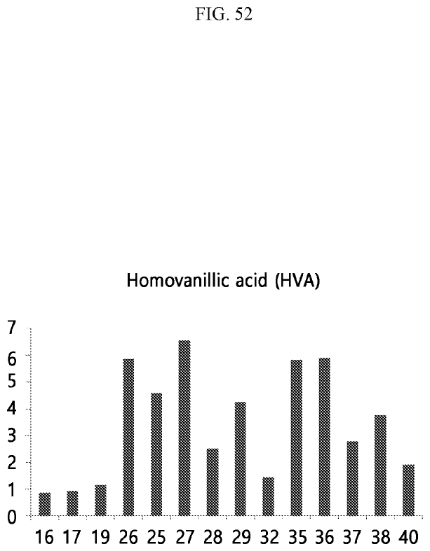

FIGS. 42 to 52 illustrates the concentration of each neurotransmitter in a plurality of positions in the human brain according to an embodiment.

The horizontal axis of the graphs illustrated in FIGS. 42 to 52 represents each region of the primate brain, and the vertical axis represents a concentration (ng/ml) at which each material is present in each region of the primate brain.

The following table shows a plurality of positions of the human brain shown by the horizontal axis.

TABLE 2

| NO. | REGION NAME |
|---|---|
| 1 | Superior frontal gyrus |
| 2 | Middle frontal gyrus |
| 3 | Inferior frontal gyrus |
| 4 | Superior temporal gyrus |
| 5 | Middle temporal gyrus |
| 6 | Inferior temporal gyrus |
| 7 | Superior parietal lobule |
| 8 | Inferior parietal lobule |
| 9 | Orbital gyrus |
| 10 | Medial occipito-temporal gyrus |
| 11 | Lateral occipito-temporal gyrus |
| 12 | Calcarine sulcus |
| 13 | Parahippocampal gyrus |
| 14 | Medial prefrontal cortex |
| 15 | Insula |
| 16 | External capsule |
| 17 | Internal capsule |
| 18 | Corpus callosum |
| 19 | Claustrum |
| 20 | Anterior Cingulate gyrus |
| 21 | Posterior Cingulate gyrus |
| 22 | Rectus gyrus |
| 23 | Cerebellar cortex |
| 24 | White mater of cerebellum |
| 25 | Caudate Nucleus |
| 26 | Lentiform Nucleus |
| 27 | Putamen |
| 28 | Globus Pallidus |
| 29 | Nucleus Accumbens |
| 30 | Amygdala |
| 31 | Thalamus |
| 32 | Hypothalamus |
| 33 | Hippocampus |
| 34 | Dentate gyrus |
| 35 | Substantia nigra_Compacta |
| 36 | Substantia nigra_Reticulata |
| 37 | Red nucleus |
| 38 | Ventral tegmental area |
| 39 | Dentate Nuclei of cerebellum |
| 40 | Raphe of midbrain |

Each identification number associated with the plurality of regions shown on the horizontal axis of FIGS. 42 to 52 represents each region in the brain shown in Table 2 above.

5.2 Anatomically Matched Human Brain and Primate Brain

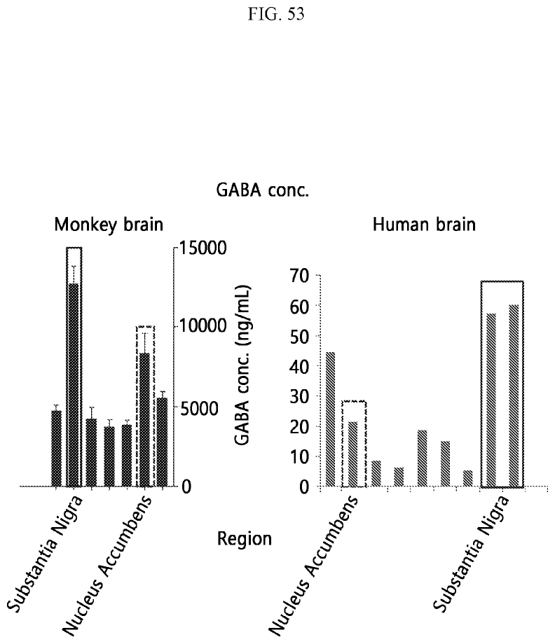

FIG. 53 is a graph showing the concentration of GABA measured in a plurality of regions of the primate brain and the human brain according to an embodiment.

According to an embodiment, the similarity between a primate brain map and a human brain map may be determined by the anatomical similarity between the primate brain and the human brain. That is, the similarity between the primate brain map and the human brain map may be matched based on the regions with similar positions occupying the entire brain when the brains of the primate and human are dissected. Accordingly, a preclinical phase which is substantially similar to the clinical phase may be provided based on the similarity between the human brain and the primate brain.

That is, when an anatomical similarity between the human brain map and the primate brain map is provided, a region in the human brain where the concentration of a specific neurotransmitter is a predetermined level or more can be matched with that of the primate brain using an anatomical similarity. Accordingly, when a sample from which the concentration of a neurotransmitter is analyzed using a sample-obtaining means in a region of the primate brain where the concentration of a specific neurotransmitter is a predetermined level or more, it is possible to provide an effect similar to analyzing the concentration of the specific neurotransmitter at a region where the concentration of the specific neurotransmitter is a predetermined level or more in the human brain.

For example, referring to FIG. 53, when attempting to analyze the concentration of GABA in the human brain, it is possible to select a region in the primate brain, which is anatomically identical to a region in the human brain where GABA is present at a concentration which is a predetermined level or more. More specifically, a region in the human brain where the concentration of GABA is highest may be Substantia Nigra. In this case, the Substantia Nigra region may be matched as a region in the primate brain, which is anatomically similar to the Substantia Nigra region among the human brain regions.

Accordingly, when a sample is obtained from the Substantia Nigra region of the primate brain using a sample-obtaining means and the concentration of GABA is analyzed from the obtained sample, an effect similar to analyzing the concentration of GABA in the Substantia Nigra region of the human brain may be provided.

For another example, referring to FIG. 53, when attempting to analyze the concentration of GABA in the human brain, due to the concentration of GABA being below a predetermined level, regions which are not appropriate for obtaining a sample may be selected by the anatomical similarity between the human brain map and the primate brain map. More specifically, the Nucleus Accumbens region where the concentration of GABA is less than a predetermined level in the human brain is a region anatomically similar to the Nucleus Accumbens region in the primate brain, but Nucleus Accumbens among the human brain regions and Nucleus Accumbens among the primate brain regions have a concentration of GABA, which is below a predetermined level, and thus may be selected as regions which are not appropriate for obtaining a sample in the primate brain.

5.3 Human Brain and Primate Brain Matched Based on Similarity in Distribution of Neurotransmitters

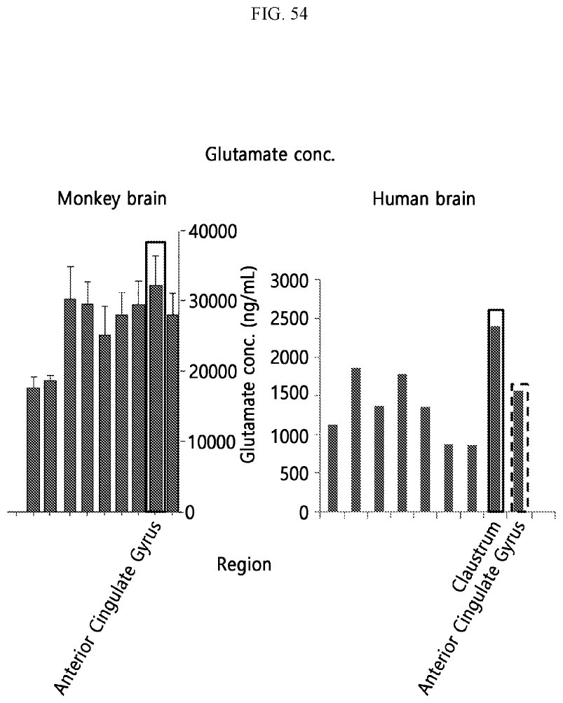

FIG. 54 is a graph showing the concentration of GABA measured in a plurality of regions of the primate brain and the human brain according to an embodiment.

According to an embodiment, the similarity between a primate brain map and a human brain map may be determined by a similarity in the concentrations of a neurotransmitter between the primate brain and the human brain. More specifically, the concentrations of a specific neurotransmitter may be similar even though the human brain and the primate brain are anatomically dissimilar. Accordingly, the concentrations of the specific neurotransmitter may be similar to each other, even at anatomically different regions in the primate brain and the human brain, and based on this, a sample obtaining position for analyzing a specific neurotransmitter may be selected.

That is, when a sample from which the concentration of a neurotransmitter is analyzed using a sample-obtaining means in a region of the primate brain where the concentration of a specific neurotransmitter is a predetermined level or more, it is possible to provide an effect similar to analyzing the concentration of the specific neurotransmitter at a region where the concentration of the specific neurotransmitter is a predetermined level or more in the human brain.

For example, referring to FIG. 54, when attempting to analyze the concentration of glutamate in the human brain, it is possible to select a region in the primate brain, which has a distribution of neurotransmitters, which is similar to a region in the human brain where glutamate is present at a concentration which is a predetermined level or more. More specifically, the Anterior Cingulate gyrus region where the concentration of glutamate is a predetermined level or more among the primate brain regions is anatomically identical to the Anterior Cingulate gyrus region among the human brain regions, but the two regions shows different glutamate concentrations, so that it may not be appropriate to match the Anterior Cingulate gyrus region among the primate brain with the Anterior Cingulate gyrus region among the human brain. In contrast, the concentration of glutamate in the Anterior Cingulate gyrus region among the primate brain regions shows a concentration distribution similar to that of the Claustrum region among the human brain regions, so that the Anterior Cingulate gyrus region among the primate brain regions and the Claustrum region among the human brain regions may be matched with each other.

Accordingly, when attempting to analyze the concentration of glutamate in the Claustrum among the human brain regions, the Anterior Cingulate gyrus region of the primate brain may be selected as a region for obtaining a sample for analyzing the concentration of glutamate.

5.4 Selection of Sample-Obtaining Region Through Similarity Between Human Brain Region and Primate Brain Region

The human brain and the primate brain may be matched in the above-described manner.

According to an embodiment, when attempting to analyze the concentration of a specific neurotransmitter in the human brain, among the regions where the human brain region and the primate brain region are matched with each other, a region in the primate brain where the concentration of the corresponding neurotransmitter is a predetermined level or more may be selected as a region for obtaining a sample.

For example, referring to previously mentioned FIG. 53, Substantia Nigra may be selected as a region of the primate brain matched to analyze the concentration of GABA in the human brain. In this case, since Substantia Nigra corresponds to a region among the primate brain regions where the concentration of GABA is a predetermined level or more, the Substantia Nigra region among the primate brain regions may be selected as a region to obtain a sample for analyzing the concentration of GAB A.

According to another embodiment, when attempting to analyze the concentration of a specific neurotransmitter in the human brain, among the regions in the primate brain where the concentration of a specific neurotransmitter is a predetermined level or more, a region which matches the human brain may be selected as a region where a sample for analyzing the concentration of the corresponding neurotransmitter is obtained.

For example, referring to previously mentioned FIG. 54, a region in the primate brain where the concentration of glutamate is a predetermined level or more may be the Anterior Cingulate gyrus region. In this case, since the Anterior Cingulate gyrus region of the primate brain and the Claustrum region of the human brain may match each other, the Anterior Cingulate gyrus region of the primate brain is may be selected as a position where a sample for analyzing glutamate is obtained.

For another example, referring to previously mentioned FIG. 54, when attempting to analyze the concentration of glutamate in the Anterior Cingulate gyrus in the human brain, the Anterior Cingulate gyrus region among the human brain regions and the Anterior Cingulate gyrus region among the primate brain regions do not match each other, and thus may not be appropriate as a position where a sample for analyzing glutamate is obtained even though the concentration of glutamate in the primate brain region is a predetermined level or more in the Anterior Cingulate gyrus region.

Experimental Method Examples

Since almost all neuropsychiatric disorders, such as degenerative neurological disorders and psychiatric disorders, are accompanied by nervous system dysregulation, it is certain that the abnormal release of the corresponding neurotransmitter is a fundamental pathological mechanism, and since drugs developed for the treatment of neuropsychiatric disorders are effective for rodents, but are not effective for humans, and thus mostly fail in the clinical phase, by evaluating the effect of drugs on fluctuations in release of neurotransmitters in primates whose nervous system is similar to that of humans, the result can be investigated as a biomarker and utilized as an efficacy evaluation tool for therapeutic agents for neuropsychiatric disorders.

Accurate identification of the causative neural circuits and restoration of specific behavioral disorders or nerve damage induced by circuit abnormalities with neural circuit regulation to enhance understanding of the etiology of mental or neurological disorders may be ideal. However, it may be difficult to confirm the circuit level by general experimental methods even in neuropsychiatric disorders or even in normal neural circuit function. Recently, optogenetic methods have made it possible to selectively regulate nerve activity, but since nerve function is regulated not only by the activity of a specific gene but also by the interaction with glial cells around the nerve, the ultimate phenotype is manifested by a combined action of neurotransmitters, neuropeptides and cytokines, so neurotransmitters, neuropeptides and cytokines should be able to be integrally analyzed, and also, since the brain consists of a number of systems that are organically linked based on a structure to maintain a physiological and pathological function, the difference between energy metabolism by a structure unit and release of neurotransmitters differs from tissue to tissue, and fluctuations in release of them will trigger the development or consequences of neuropsychiatric disorders, so that first, it is necessary to complete a neurotransmitter map which confirms the basal concentration of neurotransmitters in a normal state and confirms the organic differences between connective tissues, and based on this, a proof of concept can determine the effect of a drug by confirming the fluctuation of neurotransmitters to establish it as a biomarker and confirming the variability of neurotransmitters in tissues by therapeutic drugs.

6.1 Preparation of Experimental Animals

The nerve function is regulated by 80 billion neurons and 1000 trillion synapses present in the brain, which are regulated by neurotransmitters and neuromodulators which are released into the synapse, and to elucidate this process and mechanism that progress in the human brain, it is essential to perform an experiment which is not possible in other mammals and confirms the release regulation of neurotransmitters and metabolites in the brains of the most similar primates. Primates are a group of animals that are most similar to humans in many areas such as genetics, anatomical physiology, internal secretion, skeleton, and behavioral patterns among all existing animals, and taxonomically belong to the same order as humans, and there are 260 species of primates, which are divided into apes, old-world monkeys, new-world monkeys, and primitive monkeys, and among the old-world monkeys, rhesus monkeys and cynomolgus monkeys may be most frequently used in experiments. In the case of Europe, there are primate research facilities managed by eight countries throughout the EU, including the United Kingdom, France, Germany, the Netherlands, Italy, and the like. In particular, the United States has eight primate centers, and an average of 2,000 or more primates are being preserved per center, in Japan, the Tsukuba Primate Center for Medical Science and the Primate Research Institute of Kyoto University are leading primate research institutes, China has the largest exporter of primate resources for research in the world and the largest number of primate production facilities in the world, and is concentrating on primate research, and in Korea, the Primate Resource Center in Jeongeup currently holds about 500 animals and plans to expand the number to 3,000 animals. In addition, the Korea Institute of Toxicology, the Experimental Animal Center of the High-Tech Medical Complexes in Daegu and Osong, and the Seoul National University Hospital are breeding primates, and ORIENT BIO is building a mass breeding facility for primates in Cambodia.

In the present example, the following experiments were performed using cynomolgus monkeys among various monkeys.

A total of 10 cynomolgus monkeys (10 males, 3 to 4 kg) were purchased, then quarantined for about 1 month, and subjected to an acclimatization period for 1 month or more before the start of the test in the animal room. As an experimental group, healthy individuals with no observed symptoms affecting results were selected. During the test period, the animals were bred individually in a stainless steel breeding box (510 W×800 L×764H mm). The animals were bred under the breeding conditions of a temperature of 20 to 29° C., a humidity of 30 to 70%, a 12 hour light/dark cycle, and an illuminance of 300 to 700 Lux. About 120 g of feed a day was limitedly given in the morning and afternoon, and drinking water was given freely. All animals were managed in compliance with the guidelines of the Institutional Animal Care and Use Committee (IACUC) at the Korea Institute of Toxicology.

6.2 Separation of Monkey Brain from Body

In the present invention, brain separation and collection of brain tissue were performed according to the following steps.

1) After the monkey was made to sit in a fixed cage for monkeys, respiratory general anesthesia was induced with 4% isoflurane.

2) The anesthetized monkey was transferred to a surgical table and allowed to maintain breathing with 2% isoflurane, and it was checked whether blood pressure, heart rate, breathing, and the like were maintained within normal ranges.

3) A surgical dissector was used to make an oval incision in the skull from the frontal cortex to the occipital cortex.

4) After the dura mater was removed, cerebrospinal fluid was collected and the brain was separated from the skull.

5) The separated brain was immediately stored in a refrigerator at −70° C.

6) When the frozen brain was naturally thawed on ice and reached about 0° C., the lower part of the monkey's brain was placed on a brain matrix that was stably fixed and had regular grooves at 2 mm intervals and cut while simultaneously fitting and putting a razor in the groove, thereby making a section having a thickness of 2 mm.

7) The brain tissue separated into sections was separated at regular intervals using a tissue separation punch having a diameter of 1.37 to 2.0 mm while maintaining about 0 to 4° C. on ice, and was placed in an Eppendorf tube

8) The weight of the separated brain tissue was measured on an analytical scale, the brain tissue separated into sections was separated at regular intervals using a tissue separation punch having a diameter of 1.37 to 2.0 mm while maintaining about 0 to 4° C. on ice, and was placed in an Eppendorf tube.

9) After a brain tissue (about 20 mg) was completely homogenized with 10-fold acetonitrile (about 200 μL) and 5 μL internal standards (250 ng/mL), the brain tissue was centrifuged at 12,000 rpm for 10 minutes in a refrigerated centrifuge, and then the supernatant was isolated and put into a sample injection vial, and injected into a mass spectrometer (LC-MS/MS), thereby analyzing neurotransmitters and metabolites thereof.

6.3 Sample Administration

Venlaffaxine (Venlafaxine hydrochloride) was prepared by being dissolved in physiological saline (0.9% NaCl) at an appropriate concentration. Venlafaxine (Venlafaxine hydrochloride) is a serotonin-norepinephrine reuptake inhibitor, and is a drug used as an antidepressant. Further, donepezil (donepezil hydrochloride) was also prepared by being dissolved in physiological saline. Donepezil (Donepezil hydrochloride) is a drug used as a therapeutic agent for Alzheimer's disease.

6.4 Analytical Method (LC-MS/MS)

An analysis of neurotransmitters, which was difficult to measure by analytical methods such as general HPLC and electrochemical detectors, has now become possible with much higher sensitivity and simultaneous quantitative analysis using a mass spectrometer (LC-MS/MS). Furthermore, since the established method can simultaneously analyze not only monoamines and amino acid-based neurotransmitters but also metabolites thereof, it is also possible to evaluate the effect of the metabolic rate of neurotransmitters acting on neuropsychiatric diseases.

As the LC and MS/MS used in the present invention, an LC consisting of ACQUITY UPLC I-Class PLUS System manufactured by Waters Corporation and an MS/MS consisting of Triple Quadrupole 6500+ System manufactured by AB SCIEX were used, respectively, and as an ionization method of MS, positive and negative ions were simultaneously analyzed using an electrospray ionization (ESI) method. The ion separation method was analyzed by reverse phase liquid chromatography using an LC-MSMS equipped with a triple quadrupole mass separation tube. As the separation conditions, B was changed to a 5% composition at 1 minute and a 40% composition until 2 minutes, and B was changed to a 90% gradient composition at 2.5 minutes and the 90% gradient composition was maintained until 4.5 minutes. As the separation conditions, ACQUITY UPLC HSS T3 (2.1×100 mm, 1.8 μm, Waters) columns were used, and as a mobile phase, an aqueous solution (A) including 0.1% formic acid and 5 mM ammonium formate in HPLC water and an aqueous solution (B) including 5 mM ammonium formate in a 1:1 mixture of methanol and acetonitrile were used to maintain B starting from a 0.5% composition based on the initial 0 minute until 0.5 minute, and B was changed to a 5% composition at 1 minute and a 40% composition until 2 minutes, and B was changed to a 90% gradient composition at 2.5 minutes and the 90% gradient composition was maintained until 4.5 minutes. Stabilization was performed at the initial composition rate by flowing B at 4.6 minutes to a 0.5% composition until 7.5 minutes. The flow rate was set to 0.3 mL/min and used. The column temperature was maintained at 25° C. and 10 μl of each sample was injected thereinto. The above conditions were used in the same manner as in the following table (LC analysis conditions).

TABLE 3

| Instrument | Waters ACQUITY UPLC I-Class PLUS System | | |
|---|---|---|---|
| Column | Waters ACQUITY UPLC HSS T3 (2.1 × 100 mm, | | |
| | 1.8 μm) | | |
| Mobile phase (A) | Water (0.1% Formic acid, 5 mM Ammonium formate) | | |
| Mobile phase (B) | MeOH:Acetonitrile = 1:1 (5 mM Ammonium formate) | | |
| Gradient | Time (min) | A % | B % |
| | 0.0 | 99.5 | 0.5 |
| | 0.5 | 99.5 | 0.5 |
| | 1.0 | 95.0 | 5.0 |
| | 2.0 | 60.0 | 40.0 |
| | 2.5 | 10.0 | 90.0 |
| | 4.5 | 10.0 | 90.0 |
| | 4.6 | 99.5 | 0.5 |
| | 7.5 | 99.5 | 0.5 |

| | |
|---|---|
| Column Temp | 25° C. |
| Injection volume | 10 μl |
| Flow rate | 0.3 mL/min |

The above conditions were used in the same manner as in the following Table 4 (MS/MS analysis conditions). As a mass spectrometer, ESI was used for analysis, and nitrogen was used as a drying gas. The mass spectrometer was operated under multiple reaction monitoring (MRM), which is a multi-component simultaneous analytical method. Both positive ion and negative ion spray voltages were fixed at 4500 V. Curtain gas (CUR), Collision gas (CAD), Ion source gas 1 (GS1), and Ion source gas 2 (GS2) were maintained at 30, medium, 50, and 60, respectively, and the temperature of the ion transfer tube was fixed at 550° C. The above conditions were the same as in the following Table 4 (MS/MS analysis conditions).

TABLE 4

| Instrument | AB SCIEX Triple Quadrupole 6500+ |
|---|---|
| Ion Source Type | ESI |
| Positive Ion Spray Voltage (V) | 4500 |
| Negative Ion Spray Voltage (V) | −4500 |
| Curtain gas (CUR) | 30 |
| Collision gas(CAD) | Medium |
| Ion source gas 1(GS1) | 50 |
| Ion source gas 2(GS2) | 60 |
| Ion Transfer Tube Temp (° C.) | 550 |

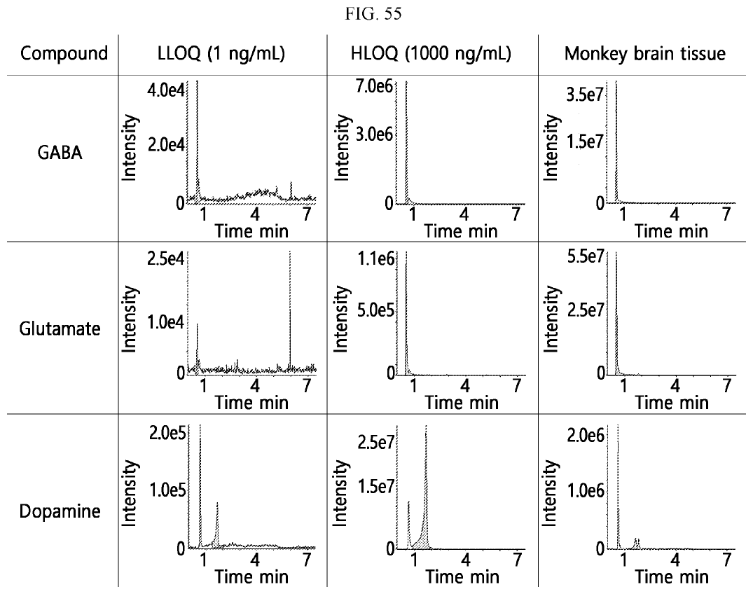

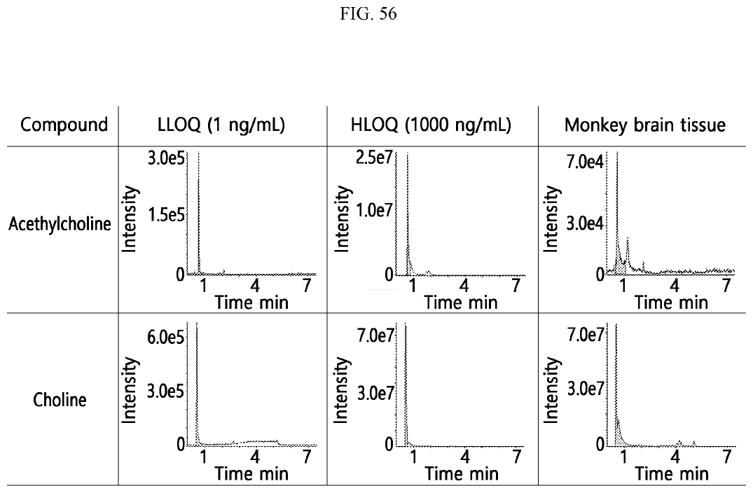

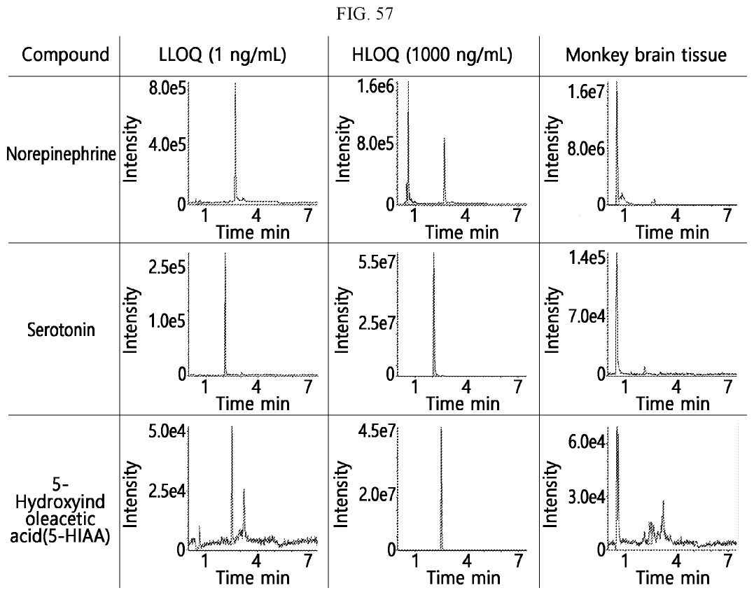

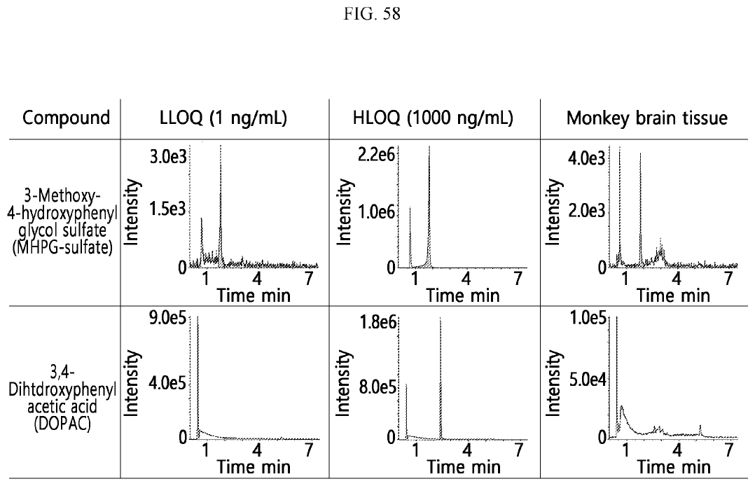

As a result of the brain neurotransmitter analysis, the chromatogram is as illustrated in FIGS. 55 to 58. The methods according to the embodiments may be implemented in the form of program instructions which can be performed through various computer means and recorded on a computer-readable medium. The computer-readable medium may include program instructions, data files, data structures, and the like, either alone or in combination. The program instructions recorded on the medium may be specially designed and configured for the embodiments, or may be known and available to those skilled in the art of computer software. Examples of computer-readable recording media include magnetic media such as hard disks, floppy disks and magnetic tapes, optical media such as CD-ROMs and DVDs, magneto-optical media such as floptical disks, and hardware devices specially configured to store and execute program instructions such as a ROM, RAM, and flash memory. Examples of program instructions include not only machine language code as produced by a compiler, but also advanced language code which can be executed by a computer using an interpreter or the like. The hardware device described above may be configured to operate as one or more software modules to perform the operation of the embodiments, and vice versa.

Although the embodiments have been described with limited embodiments and drawings as described above, those skilled in the art can make various modifications and variations from the above description. For example, even though the described techniques are performed in a different order from an order described above, and/or the described components such as systems, structures, devices, and circuits are combined in a different manner from a manner described above, or the components are replaced or substituted with other components or equivalents thereof, appropriate results can be achieved.

Therefore, other embodiments, other examples and equivalents to the appended claims also belong to the scope of the claims to be described below.

### BEST MODE OF THE INVENTION

An embodiment for practicing the present invention may include the above-described best mode for practicing the invention, and related matters are described in the best mode for practicing the invention.

1. A method for evaluating the efficacy of a drug capable of increasing or decreasing a specific neurotransmitter, wherein the method utilizes a change in a concentration of the specific neurotransmitter in a specific region of a brain as an evaluation index referring to a brain map,
the method comprises:
selecting a first region in a non-human animal's brain as a microdialysis target region;
wherein the first region corresponds to a region indicated in the brain map, where a concentration of a first neurotransmitter being increased or decreased by the drug is beyond or equal to a predetermined level;
injecting the drug into the non-human animal and confirming a change in the concentration of the first neurotransmitter at the first region before and after the injection of the drug, respectively;
wherein, the brain map is prepared by followings:
obtaining (i) a first concentration distribution for a plurality of neurotransmitters including serotonin, dopamine, GABA, glutamate, and metabolites thereof, which is obtained by performing a mass spectrometry on a sample taken from a plurality of regions of the extracted human's brain, and (ii) a second concentration distribution for a plurality of neurotransmitters including serotonin, dopamine, GABA, glutamate, and metabolites thereof, which is obtained by performing a mass spectrometry on a sample taken from a plurality of regions of the non-human animal's brain,
making the brain map using a first correlation and the second concentration distribution, wherein the first correlation includes at least 11 correlation data in which the plurality of regions of the human's brain correspond to the plurality of regions of the non-human animal's brain each other based on the similarity of the concentration distributions of the individual neurotransmitter,
when the amount of the first neurotransmitter in the first concentration distribution is beyond or equal to the predetermined level, a corresponding region is determined as a second region, and
a region on the second concentration distribution, which corresponds to the second region is determined as the first region.

2. The method of claim 1, wherein the plurality of regions of the human's brain includes at least two or more of Superior frontal gyrus, Middle frontal gyrus, Inferior frontal gyrus, Superior temporal gyrus, Middle temporal gyrus, Inferior temporal gyrus, Superior parietal lobule, Inferior parietal lobule, Orbital gyrus, Medial occipito-temporal gyrus, Lateral occipito-temporal gyrus, Calcarine sulcus, Parahippocampal gyrus, Medial prefrontal cortex, Insula, External capsule, Internal capsule, Corpus callosum, Claustrum, Anterior Cingulate gyrus, Posterior Cingulate gyrus, Rectus gyrus, Cerebellar cortex, White mater of cerebellum, Caudate Nucleus, Lentiform Nucleus, Putamen, Globus Pallidus, Nucleus Accumbens, Amygdala, Thalamus, Hypothalamus, Hippocampus, Dentate gyrus, Substantia nigra Compacta, Substantia nigra Reticulata, Red nucleus, Ventral tegmental area, Dentate Nuclei of cerebellum and Raphe of midbrain.

3. The method of claim 1, wherein the non-human animal is a primate, wherein a plurality of regions of the primate's brain includes at least two or more of Cerebellar Cortex-White Mater, Cerebellar Cortex-Gray Mater, Frontal Cortex, Occipital Cortex, Temporal Cortex, Parietal Cortex, Orbital Cortex, Visual Cortex, Superior Colliculus, Lateral Geniculate Body, Medial Geniculate Body, VTA, Substantia Nigra, Hippocampus, Posterior Cingulate Cortex, Auditory Cortex, Somatosensory Cortex, Motor Cortex, Insula, Hypothalamus, Thalamus, Perirhinal Cortex, Entorhinal Cortex, Periamygdaloid cortex, Nucleus Accumbens, Putamen, Caudate Nucleus, Anterior Cingulate Cortex and Medial PFc.

4. The method of claim 1, wherein the plurality of neurotransmitters further comprises at least one of tyramine, tryptamine, octopamine, 2-phenylalanine, aspartic acid, glutamine, 5-HIAA, norepinephrine, MHPG-sulfate, epinephrine, acetylcholine, choline, DOPAC, HVA, 3-MT, substance P, beta-endorphine, Met-enkephalin, Leu-enkephalin, dynorphin A, agmatine, spermine, spermidine, putrescine and metabolites thereof.

5. The method of claim 1, wherein the method further comprises:
confirming a change in the concentration of a second neurotransmitter in the first region, wherein the second neurotransmitter is different from the first neurotransmitter.

6. The method of claim 1, wherein the method further comprises:
selecting a third region different from the first region of the non-human animal as a microdialysis target region; and
confirming a change in the concentration of a second neurotransmitter at the third region;
wherein the second neurotransmitter is different from the first neurotransmitter capable of being increased or decreased by the drug,
the third region is determined as follows, with reference to the brain map prepared using the first concentration distribution and the second concentration distribution:
when the amount of the second neurotransmitter in the first concentration distribution is beyond or equal to the predetermined level, a corresponding region is determined as a fourth region, and
a region on the second concentration distribution, which corresponds to the fourth region is determined as the third region.

7. The method of claim 1, wherein the first correlation is obtained based on distributions of neurotransmitter concentrations in the plurality of regions which are anatomically identical between the human's brain and the non-human animal's brain.

8. The method of claim 3, wherein the primate is a monkey.

9. A method for evaluating the efficacy of a drug capable of increasing or decreasing a specific neurotransmitter referring to a brain map,
wherein the method utilizes a change in a concentration of the specific neurotransmitter in a specific region of a brain as an evaluation index referring to a brain map, and the specific region in the brain is a region in the human brain showing a difference in the concentration distribution of the specific neurotransmitter in (a) a human with any disease and (b) a human without any disease,
wherein the brain map is prepared by:
obtaining (i) a first concentration distribution for a plurality of neurotransmitters including serotonin, dopamine, GABA, glutamate, and metabolites thereof, which is obtained by performing a mass spectrometry on a sample taken from a plurality of regions of the extracted human's brain, and (ii) a second concentration distribution for a plurality of neurotransmitters including serotonin, dopamine, GABA, glutamate, and metabolites thereof, which is obtained by performing a mass spectrometry on a sample taken from a plurality of regions of the non-human animal's brain,
making the brain map using a first correlation and the second concentration distribution, wherein the first correlation includes correlation data in which the plurality of regions of the human's brain correspond to the plurality of regions of the non-human animal's brain each other based on the similarity of the concentration distributions of the individual neurotransmitter,
wherein the method comprises:
selecting a first region of the non-human animal's brain as a microdialysis target region; wherein the first region corresponds to a second region which is a specific region in the brain map, and
injecting the drug into the animal other than human and confirming a change in the concentration of the first neurotransmitter at the first region before and after the injection of the drug, respectively;
wherein the second region is determined by:
obtaining a third concentration distribution for a plurality of neurotransmitters including serotonin, dopamine, GABA, glutamate, and metabolites thereof, which is obtained by performing a mass spectrometry on a sample taken from a plurality of regions of the (a)'s brain, and
obtaining a fourth concentration distribution for a plurality of neurotransmitters including serotonin, dopamine, GABA, glutamate, and metabolites thereof, which is obtained by performing a mass spectrometry on a sample taken from a plurality of regions of the (b)'s brain, and
comparing the third concentration distribution with the fourth concentration distribution,
determining the first neurotransmitter which has the largest difference between the third concentration distribution and the fourth concentration distribution among plurality of neurotransmitters, and
determining the second region where the first neurotransmitter is distributed,
wherein the first region is determined to be a region on the second concentration distribution corresponding to a second region.

10. The method of claim 9, wherein the plurality of regions of the human's brain included at least two or more of Superior frontal gyrus, Middle frontal gyrus, Inferior frontal gyrus, Superior temporal gyrus, Middle temporal gyrus, Inferior temporal gyrus, Superior parietal lobule, Inferior parietal lobule, Orbital gyrus, Medial occipito-temporal gyrus, Lateral occipito-temporal gyrus, Calcarine sulcus, Parahippocampal gyrus, Medial prefrontal cortex, Insula, External capsule, Internal capsule, Corpus callosum, Claustrum, Anterior Cingulate gyrus, Posterior Cingulate gyrus, Rectus gyrus, Cerebellar cortex, White mater of cerebellum, Caudate Nucleus, Lentiform Nucleus, Putamen, Globus Pallidus, Nucleus Accumbens, Amygdala, Thalamus, Hypothalamus, Hippocampus, Dentate gyrus, Substantia nigra Compacta, Substantia nigra Reticulata, Red nucleus, Ventral tegmental area, Dentate Nuclei of cerebellum and Raphe of midbrain.

11. The method of claim 9, wherein the non human-animal is a primate,
wherein a plurality of regions of the primate's brain includes at least two or more of Cerebellar Cortex-White Mater, Cerebellar Cortex-Gray Mater, Frontal Cortex, Occipital Cortex, Temporal Cortex, Parietal Cortex, Orbital Cortex, Visual Cortex, Superior Colliculus, Lateral Geniculate Body, Medial Geniculate Body, VTA, Substantia Nigra, Hippocampus, Posterior Cingulate Cortex, Auditory Cortex, Somatosensory Cortex, Motor Cortex, Insula, Hypothalamus, Thalamus, Perirhinal Cortex, Entorhinal Cortex, Periamygdaloid cortex, Nucleus Accumbens, Putamen, Caudate Nucleus, Anterior Cingulate Cortex and Medial PFc.

12. The method of claim 9, wherein the plurality of neurotransmitters further comprises at least one of tyramine, tryptamine, octopamine, 2-phenylalanine, aspartic acid, glutamine, 5-HIAA, norepinephrine, MHPG-sulfate, epinephrine, acetylcholine, choline, DOPAC, HVA, 3-MT, substance P, beta-endorphine, Met-enkephalin, Leu-enkephalin, dynorphin A, agmatine, spermine, spermidine, putrescine and metabolites thereof.

13. The method of claim 9, wherein the method further comprises:
confirming a change in the concentration of a second neurotransmitter in the first region, wherein the second neurotransmitter is different from the first neurotransmitter in the first region.

14. The method of claim 9, the method further comprises:
selecting a third region different from the first region of the non-human animal as a microdialysis target region; and
confirming a change in the concentration of a second neurotransmitter at the third region;
wherein the second neurotransmitter is determined as follows:
comparing the third concentration distribution with the fourth concentration distribution, and determining the second neurotransmitter which is a different from the first neurotransmitter among the plurality of neurotransmitters showing a difference on the comparison,
wherein the third region is determined as follows, with reference to the brain map prepared using the first concentration distribution and the second concentration distribution:
determining a region where the second neurotransmitter is distributed as a fourth region, and
determining a region on the second concentration distribution, which corresponds to the fourth region is determined as the third region.

15. The method of claim 9, wherein the first correlation is obtained based on distributions of neurotransmitter concentrations in the plurality of regions which are anatomically identical between the human's brain and the non-human animal's brain.

16. The method of claim 11, wherein the primate is a monkey.

17. The method of claim 1, wherein the method further comprises:
fixing a microdialysis probe in order to obtain a microdialysis sample from the target region after selecting the first region of an non-human animal's brain as a microdialysis target region;
wherein the fixing a microdialysis probe is performed by:
removing a moisture on a surface of the brain;
placing a mesh on the surface of the brain; wherein the mesh comprises a wire dividing the surface of the brain to a predetermined size and a plurality of cavities generated according to the division, and the mesh is to prevent the microdialysis probe from falling off,
inserting a microdialysis probe into at least one of the plurality of cavities of the mesh such that at least a portion of the microdialysis probe is inserted into the non-human animal's brain; and
adhering the microdialysis probe to a predetermined part including a position where the microdialysis probe is inserted using an adhesive in order to fix the microdialysis probe to the non-human animal's brain.
